(19) United States
(12) Patent Application Publication
(10) Pub. No.: US 2011/0264086 A1
(43) Pub. Date: Oct. 27, 2011

(54) RENAL ARTERY DENERVATION APPARATUS EMPLOYING HELICAL SHAPING ARRANGEMENT

(76) Inventor: Frank Ingle, Palo Alto, CA (US)

(21) Appl. No.: 13/087,163

(22) Filed: Apr. 14, 2011

Related U.S. Application Data
(60) Provisional application No. 61/324,165, filed on Apr. 14, 2010.

(51) Int. Cl. A61B 18/18

(52) U.S. Cl. 606/33

(57) ABSTRACT

Devices, systems, and methods provide for renal sympathetic nerve activity modification and termination. Apparatuses are configured for intravascular delivery of a denervation therapy to a renal artery of a patient, and preferably create a lesion or lesions that define a pattern that completes at least one revolution of the renal artery. Various denervation therapy elements may be employed, including a cryotherapy arrangement, a drug eluting arrangement, an RF ablation arrangement, an ultrasonic ablation catheter, a laser ablation catheter, a microwave ablation catheter, or a combination of these therapy elements.

### RELATED APPLICATIONS

This application claims the benefit of Provisional Patent Application Ser. No. 61/324,165 filed on Apr. 14, 2010, to which priority is claimed pursuant to 35 U.S.C. §119(e) and which is hereby incorporated herein by reference in its entirety.

### TECHNICAL FIELD

The present invention is related to systems and methods for improving cardiac and/or renal function through neuromodulation, including disruption and termination of renal sympathetic nerve activity.

### BACKGROUND

The kidneys are instrumental in a number of body processes, including blood filtration, regulation of fluid balance, blood pressure control, electrolyte balance, and hormone production. One primary function of the kidneys is to remove toxins, mineral salts, and water from the blood to form urine. The kidneys receive about 20-25% of cardiac output through the renal arteries that branch left and right from the abdominal aorta, entering each kidney at the concave surface of the kidneys, the renal hilum.

Blood flows into the kidneys through the renal artery and the afferent arteriole, entering the filtration portion of the kidney, the renal corpuscle. The renal corpuscle is composed of the glomerulus, a thicket of capillaries, surrounded by a fluid-filled, cup-like sac called Bowman's capsule. Solutes in the blood are filtered through the very thin capillary walls of the glomerulus due to the pressure gradient that exists between the blood in the capillaries and the fluid in the Bowman's capsule. The pressure gradient is controlled by the contraction or dilation of the arterioles. After filtration occurs, the filtered blood moves through the efferent arteriole and the peritubular capillaries, converging in the interlobular veins, and finally exiting the kidney through the renal vein.

Particles and fluid filtered from the blood move from the Bowman's capsule through a number of tubules to a collecting duct. Urine is formed in the collecting duct and then exits through the ureter and bladder. The tubules are surrounded by the peritubular capillaries (containing the filtered blood). As the filtrate moves through the tubules and toward the collecting duct, nutrients, water, and electrolytes, such as sodium and chloride, are reabsorbed into the blood.

The kidneys are innervated by the renal plexus which emanates primarily from the aorticorenal ganglion. Renal ganglia are formed by the nerves of the renal plexus as the nerves follow along the course of the renal artery and into the kidney. The renal nerves are part of the autonomic nervous system which includes sympathetic and parasympathetic components. The sympathetic nervous system is known to be the system that provides the bodies “fight or flight” response, whereas the parasympathetic nervous system provides the “rest and digest” response. Stimulation of sympathetic nerve activity triggers the sympathetic response which causes the kidneys to increase production of hormones that increase vasoconstriction and fluid retention. This process is referred to as the renin-angiotensin-aldosterone-system (RAAS) response to increased renal sympathetic nerve activity.

In response to a reduction in blood volume, the kidneys secrete renin, which stimulates the production of angiotensin. Angiotensin causes blood vessels to constrict, resulting in increased blood pressure, and also stimulates the secretion of the hormone aldosterone from the adrenal cortex. Aldosterone causes the tubules of the kidneys to increase the reabsorption of sodium and water, which increases the volume of fluid in the body and blood pressure.

Congestive heart failure (CHF) is a condition that has been linked to kidney function. CHF occurs when the heart is unable to pump blood effectively throughout the body. When blood flow drops, renal function degrades because of insufficient perfusion of the blood within the renal corpuscles. The decreased blood flow to the kidneys triggers an increase in sympathetic nervous system activity (i.e., the RAAS becomes too active) that causes the kidneys to secrete hormones that increase fluid retention and vasorestriction. Fluid retention and vasorestriction in turn increases the peripheral resistance of the circulatory system, placing an even greater load on the heart, which diminishes blood flow further. If the deterioration in cardiac and renal functioning continues, eventually the body becomes overwhelmed, and an episode of heart failure decompensation occurs, often leading to hospitalization of the patient.

Hypertension is a chronic medical condition in which the blood pressure is elevated. Persistent hypertension is a significant risk factor associated with a variety of adverse medical conditions, including heart attacks, heart failure, arterial aneurysms, and strokes. Persistent hypertension is a leading cause of chronic renal failure. Hyperactivity of the sympathetic nervous system serving the kidneys is associated with hypertension and its progression. Deactivation of nerves in the kidneys via renal denervation can reduce blood pressure, and may be a viable treatment option for many patients with hypertension who do not respond to conventional drugs.

### SUMMARY

Devices, systems, and methods of the present invention are directed to modifying renal sympathetic nerve activity. Embodiments of the present invention are directed to an apparatus for intravascular delivery of a denervation therapy to a renal artery of a patient. According to various embodiments, a renal denervation therapy apparatus includes an elongated guide rail comprising a proximal end, a distal end, and a length sufficient to access at least the renal artery from a location external of the patient. A helical section is provided at the distal end of the guide rail. The helical section has a diameter about equal to a diameter of the renal artery. A treatment apparatus has a length sufficient to access at least the renal artery from a location external of the patient and a longitudinal channel configured to receive the elongated guide rail. The treatment apparatus comprises a treatment element configured to deliver denervation therapy to the renal artery.

In particular, longitudinal displacement of the treatment apparatus relative to the helical section of the guide rail urges the treatment element into contact with an inner wall of the renal artery and to follow a generally helical path along the renal artery's inner wall for denervating a spiral shaped region of the renal artery. The treatment element may include at least one of a cryotherapy arrangement, a drug eluting arrangement (e.g., applicator or injector), an RF ablation arrangement, an ultrasonic ablation catheter, a laser ablation catheter, and a microwave ablation catheter.

In accordance with other embodiments, an apparatus for intravascular delivery of a denervation therapy to a renal artery includes a treatment catheter comprising a proximal end, a distal end, and a length sufficient to access at least the renal artery from a location external of the patient. A treatment section is provided at a distal end of the treatment catheter. The treatment section is configured for multi-planar flexing and to deliver denervation therapy to the renal artery. The apparatus further includes a balloon catheter comprising a shaft having a lumen arrangement, a proximal end, a distal end, and a length sufficient to access at least the renal artery from a location external of the patient. The balloon catheter includes an elongated balloon disposed at the distal end of the shaft and fluidly coupled to the lumen arrangement. The elongated balloon is coupled to the distal end of the treatment catheter and arranged to complete at least one revolution of the treatment catheter's distal end. The balloon is configured to contort the treatment section into a generally helical shape when inflated, such that portions of the treatment section contact regions of an inner wall of the renal artery.

According to further embodiments, an apparatus for intravascular delivery of RF denervation therapy to a renal artery includes a treatment catheter comprising a proximal end, a distal end, and a length sufficient to access at least the renal artery from a location external of the patient. A treatment element comprising a braid member is provided at a distal end of the treatment catheter and dimensioned for deployment within the renal artery. The braid member comprises a material having a resiliency sufficient to facilitate deployment of the braid member into the renal artery from the abdominal aorta, a proximal end, a distal end, a length, and a diameter. An electrically conductive pattern is provided on the braid member having a substantially helical shape that completes at least one revolution of the braid member. The electrically conductive pattern is configured to electrically couple with a radiofrequency generator. The braid member includes insulating portions defining regions of the braid member devoid of the electrically conductive pattern.

The braid member is configured to decrease in length and increase in diameter in response to axial compression, and to increase in length and decrease in diameter in response to axial tensioning or relaxation. An actuator is coupled to the braid member and actuatable at the proximal end of the treatment catheter. The actuator is coupled to at least one of the proximal and distal ends of the braid member and configured to selectively extend and compress the braid member longitudinally. The electrically conductive pattern of the braid member is urged towards and away from an inner wall of the renal artery in response to braid member compression and relaxation, respectively. Denervation therapy delivery to the renal artery is commenced with the braid member in compression and by energizing the electrically conductive pattern by the radiofrequency generator.

One or more sensors can be provided at or coupled to the braid. Suitable sensors include one or both of temperature and impedance sensors. The radiofrequency generator may be configured to automatically control power delivery to the braid in response to a signal produced by the one or more sensors during denervation therapy delivery. The braid material may comprise a plurality of voids that define a perfusion arrangement which facilitates arterial blood flow through the braid for cooling an inner wall of the renal artery.

In accordance with some embodiments, a catheter comprising a proximal end, a distal end, and a length sufficient to access at least a renal artery relative to a percutaneous access location of the patient. A balloon is disposed at the distal end of the catheter and fluidly coupled to a lumen of the catheter. The balloon is configured for deployment within the renal artery and to receive a thermal transfer fluid via the lumen. A braid is provided on a surface of the balloon and comprises a resilient material. The braid further comprises an electrically conductive pattern having a substantially helical shape that completes at least one revolution of the braid. The electrically conductive pattern is configured to electrically couple with a radiofrequency generator for delivering renal denervation therapy. Insulating portions define regions of the braid devoid of the electrically conductive pattern.

Inflation of the balloon causes the diameter of the braid to increase and the length of the braid to decrease. Deflation of the balloon causes the diameter of the braid to decrease and the length of the braid to increase. The balloon may incorporate a circulation arrangement through which a thermal transfer fluid can circulate for cooling the inner wall of the renal artery during delivery of renal denervation therapy.

According to other embodiments, a method involves extending a braid disposed at a distal end of a catheter longitudinally for deployment of the braid within a renal artery of a patient. The method also involves compressing the braid longitudinally so that an electrically conductive pattern of the braid is urged towards an inner wall of the renal artery, and energizing the electrically conductive pattern to create a lesion in the artery having a substantially spiral shape. The method further involves cooling the braid while energizing the electrically conductive pattern to cool the inner wall of the artery, and extending the braid longitudinally subsequent to energizing the electrically conductive pattern for removal of the braid from the patient's renal artery.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### DESCRIPTION OF THE DRAWINGS

FIG. 1 is an illustration of a right kidney and renal vasculature including a renal artery branching laterally from the abdominal aorta;

FIGS. 2A and 2B illustrate sympathetic innervation of the renal artery;

FIG. 3A illustrates various tissue layers of the wall of the renal artery;

FIGS. 3B and 3C illustrate a portion of a renal nerve;

FIGS. 4A, 4B, and 5 illustrate a denervation therapy apparatus employing a treatment catheter and helical shaping arrangement configured for deployment within a renal artery in accordance with embodiments of the present invention;

FIGS. 6A and 6B illustrate a denervation therapy apparatus employing a treatment catheter, helical shaping member, and balloon arrangement configured for deployment within a renal artery in accordance with embodiments of the present invention;

FIG. 7 shows a cross-section of components of a denervation therapy apparatus according to the embodiment illustrated in FIGS. 6A and 6B;

FIGS. 8A-8B and 9A-9B illustrate various embodiments of a treatment catheter and helical shaping arrangement implemented in accordance with the present invention;

FIG. 10 illustrates an embodiment of a treatment element and helical shaping arrangement implemented in accordance with the present invention;

FIG. 11 illustrates a treatment element and inflatable helical shaping arrangement implemented in accordance with embodiments of the present invention;

FIG. 12 illustrates a treatment element and inflatable helical shaping arrangement implemented in accordance with other embodiments of the present invention;

FIGS. 13-16 illustrate a treatment element and inflatable helical shaping arrangement implemented in accordance with embodiments of the present invention;

FIGS. 17A-17C are cross-sections of a distal portion of a treatment catheter apparatus in accordance with various embodiments of the present invention;

FIGS. 18A-8B, 19, and 20 illustrate various configurations of a braid member provided on a treatment catheter and having an electrically conductive pattern, the braid member configured to deform in the manner of a Chinese handcuff in accordance with embodiments of the present invention;

FIG. 21 illustrate a treatment catheter comprising a multiplicity of braid members of the type shown in FIGS. 18A-8B, 19, and 20 in accordance with embodiments of the present invention;

FIGS. 22A and 22B show a braid member having an electrically conductive pattern provided over a balloon of a treatment catheter in accordance embodiments of the invention;

FIGS. 22C and 22D show details of a braid member having an electrically conductive pattern bonded to a balloon of a treatment catheter in accordance embodiments of the invention;

FIG. 23A shows a representative embodiment of a radiofrequency (RF) renal therapy apparatus in accordance with embodiments of the present invention;

FIG. 23B shows a cross-section of a lumen arrangement of a treatment catheter apparatus in accordance with embodiments of the present invention.

FIG. 24 illustrates a portion of the treatment catheter that incorporates a hinge mechanism in accordance with embodiments of the invention; and

FIGS. 25-28 show a series of views of a treatment catheter implemented in accordance with embodiments of the present invention at different states of deployment within aortal and renal vasculature of a patient.

While the invention is amenable to various modifications and alternative forms, specifics thereof have been shown by way of example in the drawings and will be described in detail. It is to be understood, however, that the intention is not to limit the invention to the particular embodiments described. On the contrary, the intention is to cover all modifications, equivalents, and alternatives falling within the scope of the invention as defined by the appended claims.

### DETAILED DESCRIPTION

In the following description, references are made to the accompanying drawings which illustrate various embodiments of the invention. It is to be understood that other embodiments may be utilized, and structural and functional changes may be made to these embodiments without departing from the scope of the present invention.

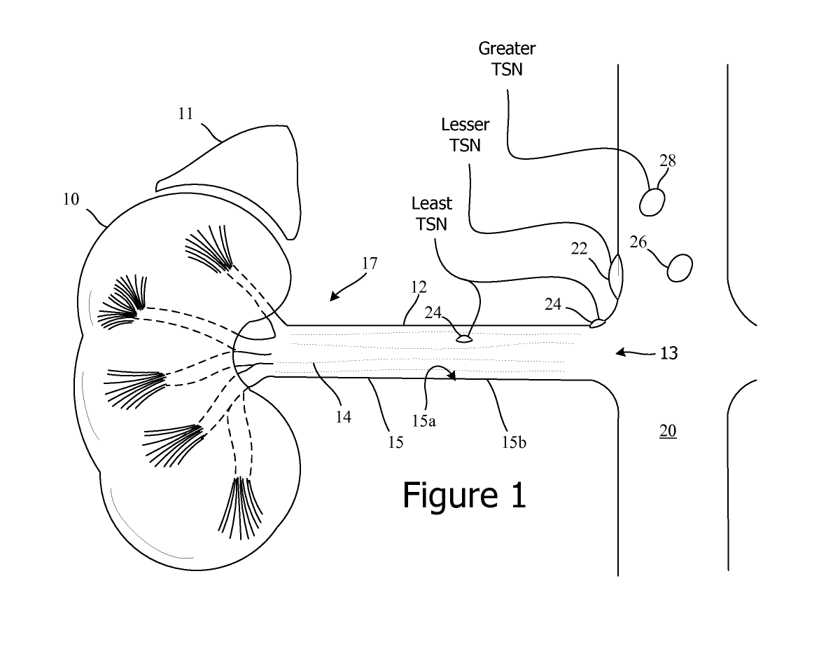

FIG. 1 is an illustration of a right kidney 10 and renal vasculature including a renal artery 12 branching laterally from the abdominal aorta 20. In FIG. 1, only the right kidney 10 is shown for purposes of simplicity of explanation, but reference will be made herein to both right and left kidneys and associated renal vasculature and nervous system structures, all of which are contemplated within the context of embodiments of the present invention. The renal artery 12 is purposefully shown to be disproportionately larger than the right kidney 10 and abdominal aorta 20 in order to facilitate discussion of various features and embodiments of the present disclosure.

The right and left kidneys are supplied with blood from the right and left renal arteries that branch from respective right and left lateral surfaces of the abdominal aorta 20. Each of the right and left renal arteries is directed across the crus of the diaphragm, so as to form nearly a right angle with the abdominal aorta 20. The right and left renal arteries extend generally from the abdominal aorta 20 to respective renal sinuses proximate the hilum 17 of the kidneys, and branch into segmental arteries and then interlobular arteries within the kidney 10. The interlobular arteries radiate outward, penetrating the renal capsule and extending through the renal columns between the renal pyramids. Typically, the kidneys receive about 20% of total cardiac output which, for normal persons, represents about 1200 mL of blood flow through the kidneys per minute.

The primary function of the kidneys is to maintain water and electrolyte balance for the body by controlling the production and concentration of urine. In producing urine, the kidneys excrete wastes such as urea and ammonium. The kidneys also control reabsorption of glucose and amino acids, and are important in the production of hormones including vitamin D, renin and erythropoietin.

An important secondary function of the kidneys is to control metabolic homeostasis of the body. Controlling hemostatic functions include regulating electrolytes, acid-base balance, and blood pressure. For example, the kidneys are responsible for regulating blood volume and pressure by adjusting volume of water lost in the urine and releasing erythropoietin and renin, for example. The kidneys also regulate plasma ion concentrations (e.g., sodium, potassium, chloride ions, and calcium ion levels) by controlling the quantities lost in the urine and the synthesis of calcitriol. Other hemostatic functions controlled by the kidneys include stabilizing blood pH by controlling loss of hydrogen and bicarbonate ions in the urine, conserving valuable nutrients by preventing their excretion, and assisting the liver with detoxification.

Also shown in FIG. 1 is the right suprarenal gland 11, commonly referred to as the right adrenal gland. The suprarenal gland 11 is a star-shaped endocrine gland that rests on top of the kidney 10. The primary function of the suprarenal glands (left and right) is to regulate the stress response of the body through the synthesis of corticosteroids and catecholamines, including cortisol and adrenaline (epinephrine), respectively. Encompassing the kidneys 10, suprarenal glands 11, renal vessels 12, and adjacent perirenal fat is the renal fascia, e.g., Gerota's fascia, (not shown), which is a fascial pouch derived from extraperitoneal connective tissue.

The autonomic nervous system of the body controls involuntary actions of the smooth muscles in blood vessels, the digestive system, heart, and glands. The autonomic nervous system is divided into the sympathetic nervous system and the parasympathetic nervous system. In general terms, the parasympathetic nervous system prepares the body for rest by lowering heart rate, lowering blood pressure, and stimulating digestion. The sympathetic nervous system effectuates the body's fight-or-flight response by increasing heart rate, increasing blood pressure, and increasing metabolism.

In the autonomic nervous system, fibers originating from the central nervous system and extending to the various ganglia are referred to as preganglionic fibers, while those extending from the ganglia to the effector organ are referred to as postganglionic fibers. Activation of the sympathetic nervous system is effected through the release of adrenaline (epinephrine) and to a lesser extent norepinephrine from the suprarenal glands 11. This release of adrenaline is triggered by the neurotransmitter acetylcholine released from preganglionic sympathetic nerves.

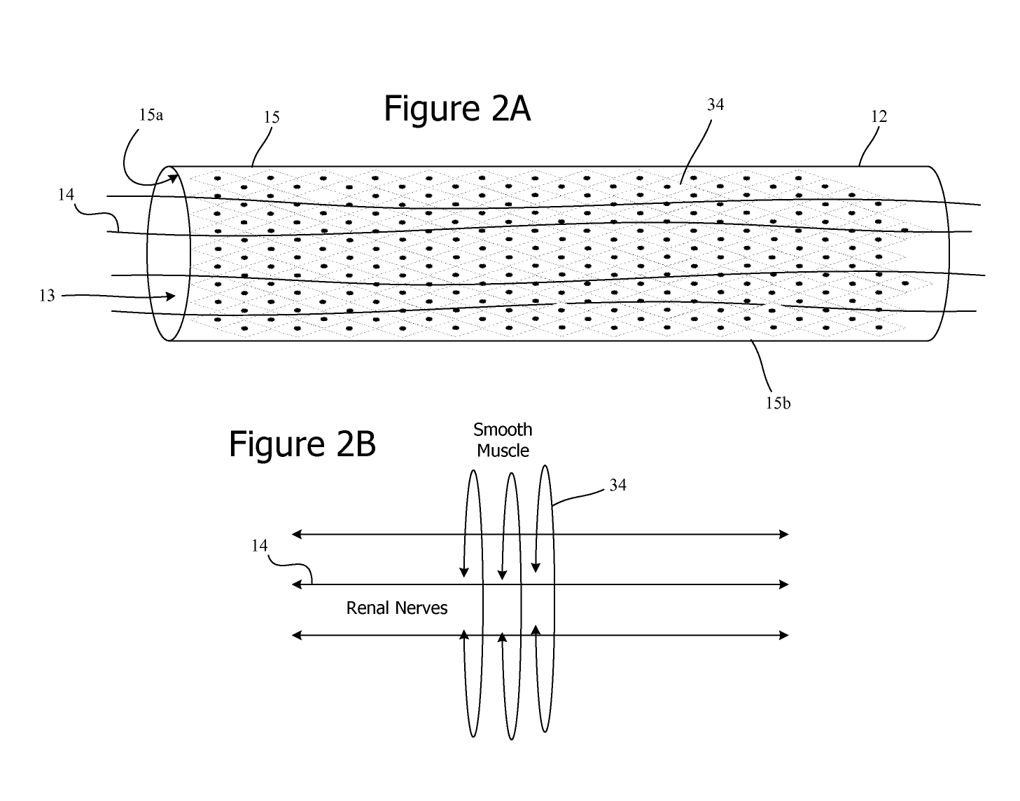

The kidneys and ureters (not shown) are innervated by the renal nerves 14. FIGS. 1 and 2A-2B illustrate sympathetic innervation of the renal vasculature, primarily innervation of the renal artery 12. The primary functions of sympathetic innervation of the renal vasculature include regulation of renal blood flow and pressure, stimulation of renin release, and direct stimulation of water and sodium ion reabsorption.

Most of the nerves innervating the renal vasculature are sympathetic postganglionic fibers arising from the superior mesenteric ganglion 26. The renal nerves 14 extend generally axially along the renal arteries 12, enter the kidneys 10 at the hilum 17, follow the branches of the renal arteries 12 within the kidney 10, and extend to individual nephrons. Other renal ganglia, such as the renal ganglia 24, superior mesenteric ganglion 26, the left and right aorticorenal ganglia 22, and celiac ganglia 28 also innervate the renal vasculature. The celiac ganglion 28 is joined by the greater thoracic splanchnic nerve (greater TSN). The aorticorenal ganglia 26 is joined by the lesser thoracic splanchnic nerve (lesser TSN) and innervates the greater part of the renal plexus.

Sympathetic signals to the kidney 10 are communicated via innervated renal vasculature that originates primarily at spinal segments T10-T12 and L1. Parasympathetic signals originate primarily at spinal segments S2-S4 and from the medulla oblongata of the lower brain. Sympathetic nerve traffic travels through the sympathetic trunk ganglia, where some may synapse, while others synapse at the aorticorenal ganglion 22 (via the lesser thoracic splanchnic nerve, i.e., lesser TSN) and the renal ganglion 24 (via the least thoracic splanchnic nerve, i.e., least TSN). The postsynaptic sympathetic signals then travel along nerves 14 of the renal artery 12 to the kidney 10. Presynaptic parasympathetic signals travel to sites near the kidney 10 before they synapse on or near the kidney 10.

With particular reference to FIG. 2A, the renal artery 12, as with most arteries and arterioles, is lined with smooth muscle 34 that controls the diameter of the renal artery lumen 13. Smooth muscle, in general, is an involuntary non-striated muscle found within the media layer of large and small arteries and veins, as well as various organs. The glomeruli of the kidneys, for example, contain a smooth muscle-like cell called the mesangial cell. Smooth muscle is fundamentally different from skeletal muscle and cardiac muscle in terms of structure, function, excitation-contraction coupling, and mechanism of contraction.

Smooth muscle cells can be stimulated to contract or relax by the autonomic nervous system, but can also react on stimuli from neighboring cells and in response to hormones and blood borne electrolytes and agents (e.g., vasodilators or vasoconstrictors). Specialized smooth muscle cells within the afferent arteriole of the juxtaglomerular apparatus of kidney 10, for example, produces renin which activates the angiotension II system.

The renal nerves 14 innervate the smooth muscle 34 of the renal artery wall 15 and extend lengthwise in a generally axial or longitudinal manner along the renal artery wall 15. The smooth muscle 34 surrounds the renal artery circumferentially, and extends lengthwise in a direction generally transverse to the longitudinal orientation of the renal nerves 14, as is depicted in FIG. 2B.

The smooth muscle 34 of the renal artery 12 is under involuntary control of the autonomic nervous system. An increase in sympathetic activity, for example, tends to contract the smooth muscle 34, which reduces the diameter of the renal artery lumen 13 and decreases blood perfusion. A decrease in sympathetic activity tends to cause the smooth muscle 34 to relax, resulting in vessel dilation and an increase in the renal artery lumen diameter and blood perfusion. Conversely, increased parasympathetic activity tends to relax the smooth muscle 34, while decreased parasympathetic activity tends to cause smooth muscle contraction.

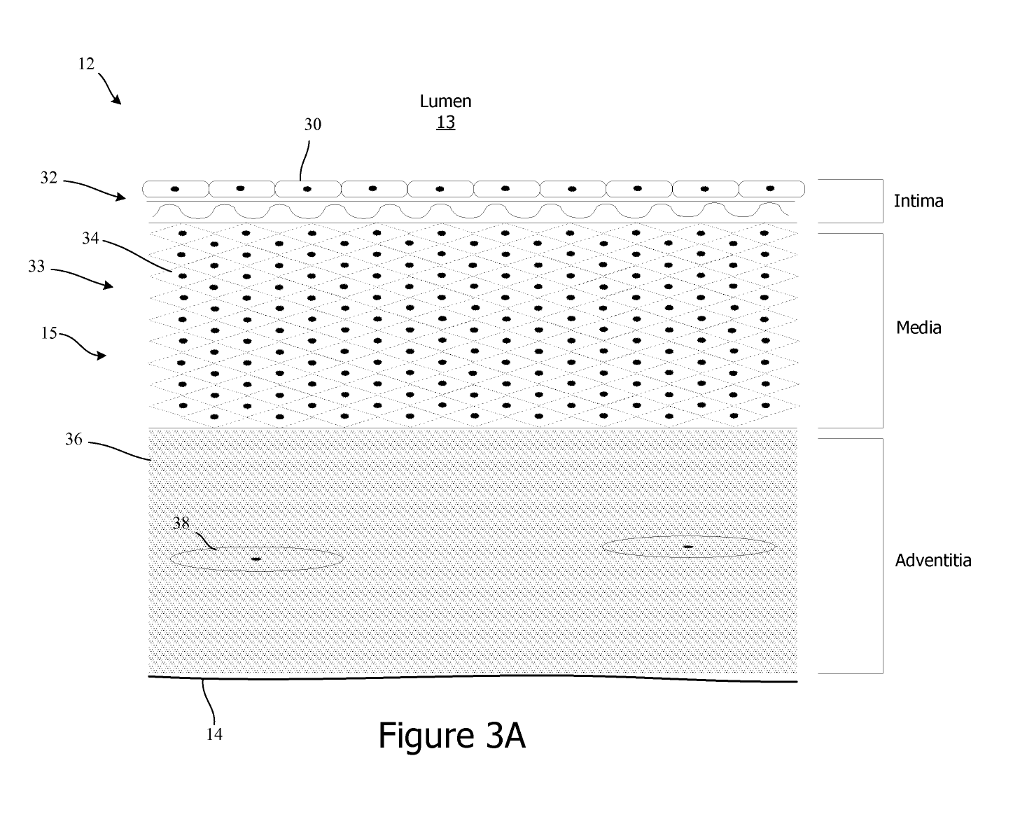

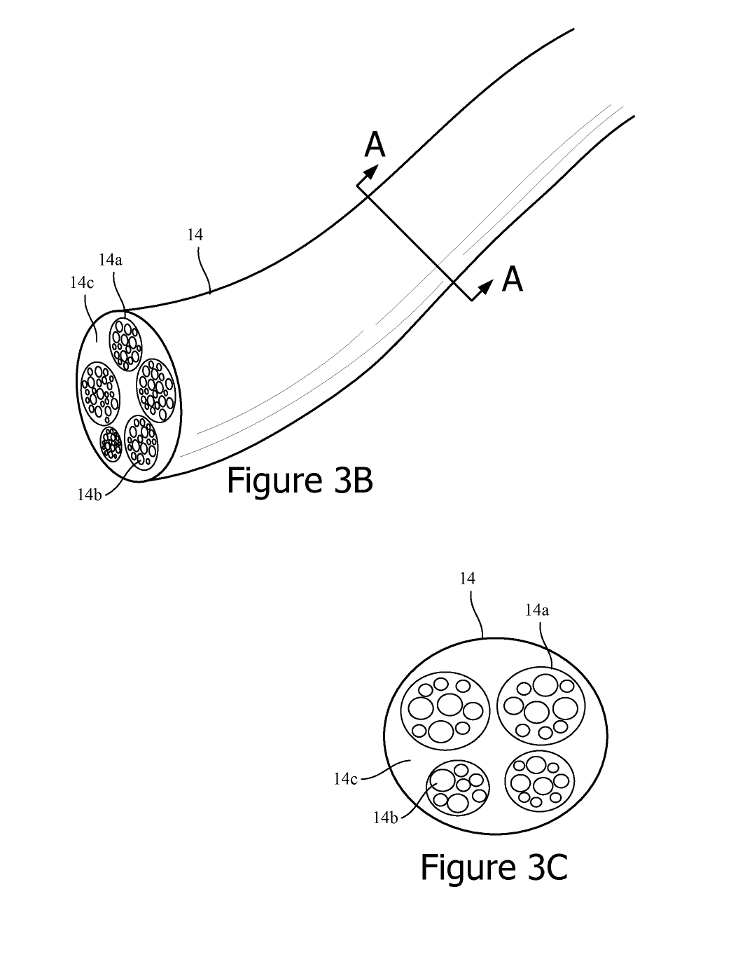

FIG. 3A shows a segment of a longitudinal cross-section through a renal artery, and illustrates various tissue layers of the wall 15 of the renal artery 12. The innermost layer of the renal artery 12 is the endothelium 30, which is the innermost layer of the intima 32 and is supported by an internal elastic membrane. The endothelium 30 is a single layer of cells that contacts the blood flowing though the vessel lumen 13. Endothelium cells are typically polygonal, oval, or fusiform, and have very distinct round or oval nuclei. Cells of the endothelium 30 are involved in several vascular functions, including control of blood pressure by way of vasoconstriction and vasodilation, blood clotting, and acting as a barrier layer between contents within the lumen 13 and surrounding tissue, such as the membrane of the intima 32 separating the intima 32 from the media 34, and the adventitia 36. The membrane or maceration of the intima 32 is a fine, transparent, colorless structure which is highly elastic, and commonly has a longitudinal corrugated pattern.

Adjacent the intima 32 is the media 33, which is the middle layer of the renal artery 12. The media is made up of smooth muscle 34 and elastic tissue. The media 33 can be readily identified by its color and by the transverse arrangement of its fibers. More particularly, the media 33 consists principally of bundles of smooth muscle fibers 34 arranged in a thin plate-like manner or lamellae and disposed circularly around the arterial wall 15. The outermost layer of the renal artery wall 15 is the adventitia 36, which is made up of connective tissue. The adventitia 36 includes fibroblast cells 38 that play an important role in wound healing. A renal nerve 14 is shown proximate the adventitia 36 and extending longitudinally along the renal artery 12. The main trunk of the renal nerves 14 generally lies in or on the adventitia of the renal artery, with certain branches coursing into the media to enervate the renal artery smooth muscle.

Embodiments of the present invention are directed to apparatuses and methods for delivering denervation therapy to a renal artery in order to modify, disrupt, or terminate renal sympathetic nerve activity. Embodiments are directed to apparatuses and methods for delivering denervation therapy to a renal artery in accordance with a predefined helical pattern. Embodiments are further directed to apparatuses and methods for forcing a denervation therapy apparatus to assume a predefined helical shape for or during renal artery denervation therapy. Preferred embodiments are those that deliver denervation therapy to a renal artery in accordance with a predefined helical pattern which irreversibly terminates renal sympathetic nerve activity.

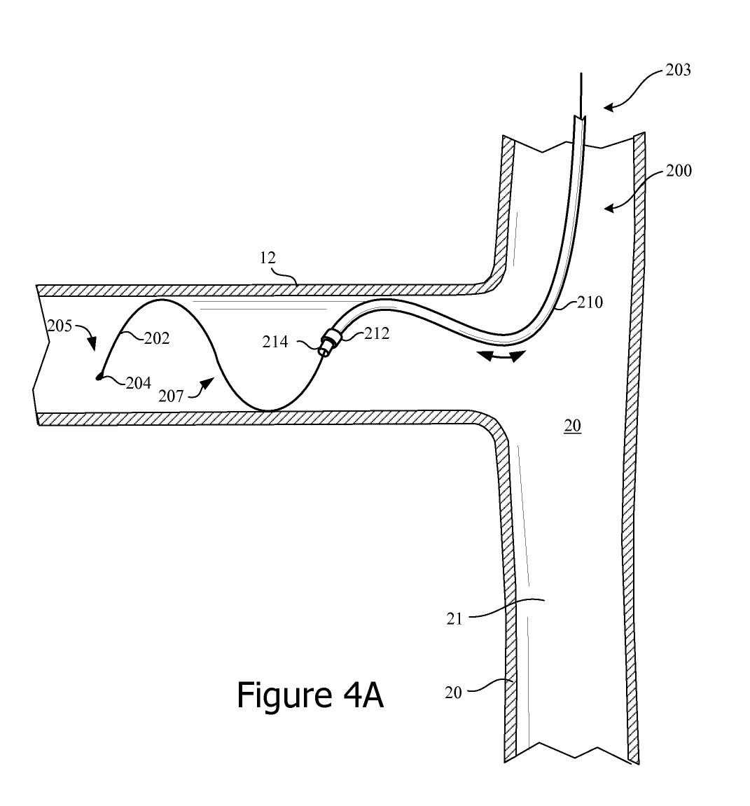

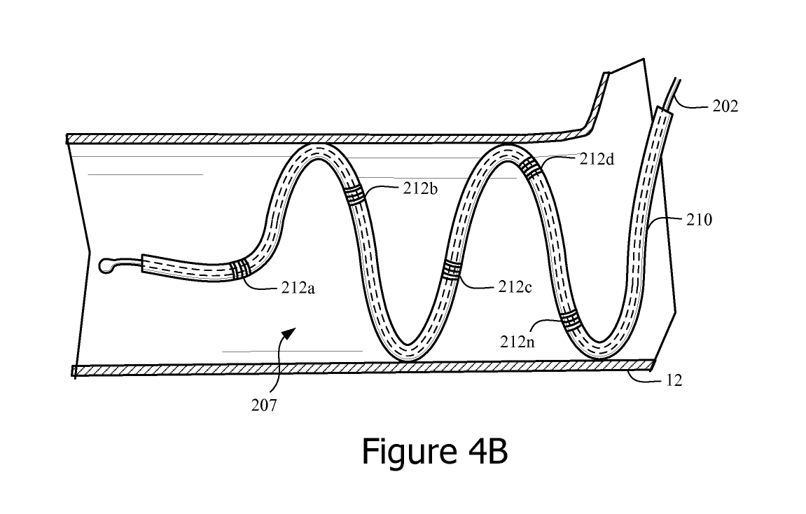

A representative embodiment of a denervation therapy apparatus employing a helical shaping arrangement for modifying, disrupting, or terminating renal sympathetic nerve activity in accordance with the present invention is shown in FIG. 4A. FIG. 4A illustrates a denervation therapy apparatus 200 configured for deployment within a renal artery 12 of a patient. The denervation therapy apparatus 200 shown in FIG. 4A includes a guide rail 202 and a treatment catheter 210. The guide rail 202 and treatment catheter 210 are configured to facilitate longitudinal displacement of the treatment catheter 210 along a generally helical path within the renal artery 12. More particularly, the guide rail 202 and treatment catheter 210 are configured to facilitate longitudinal displacement of the treatment catheter 210 along a generally helical path within the renal artery 12 that completes at least one turn or revolution of the guide rail 202.

The denervation therapy apparatus 200 shown in FIG. 4A includes a guide rail 202 having a proximal end 203 and a distal end 205. The distal end 205 preferably includes an atraumatic distal tip 204. The guide rail 202 preferably has a length that is sufficient to access at least the renal artery 12 from a location external of the patient. The proximal end 203 preferably includes, or is coupled to, a proximal control mechanism that facilitates physician manipulation of the guide rail 202.

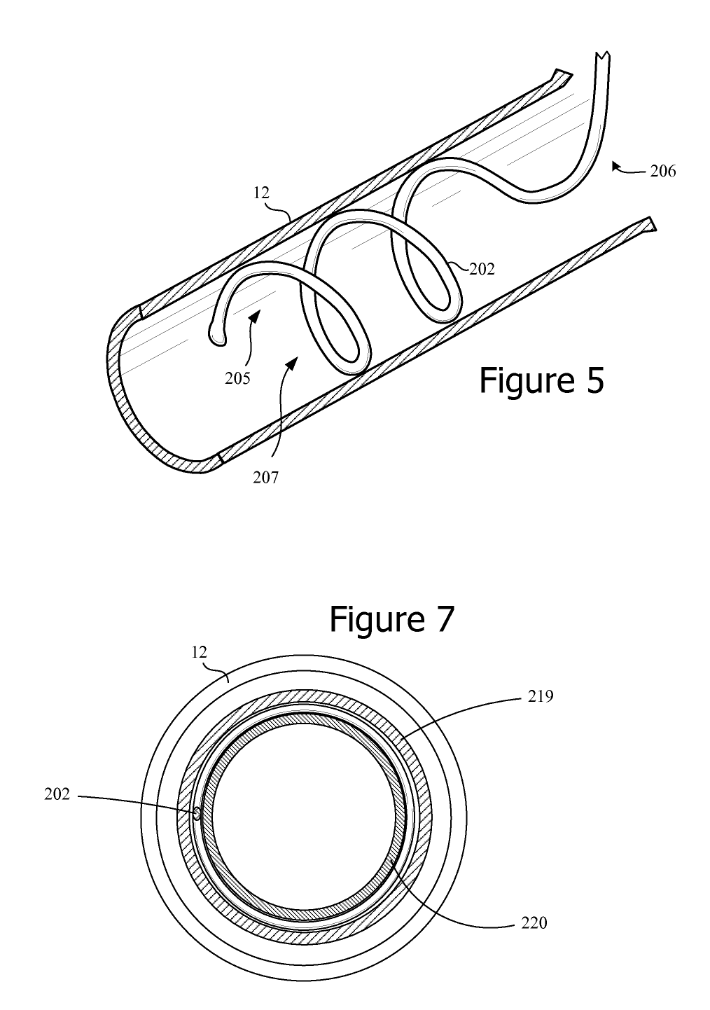

The distal end 205 of the guide rail 202 includes a helical or spiral section 207. In some embodiments, the guide rail 202 has a diameter about equal to a diameter of the renal artery 12 when in a deployed configuration. In a fully deployed configuration, as is best shown in FIG. 5, the helical section 207 is dimensioned to contact at least some regions of the inner wall of the renal artery 12, and is sufficiently resilient to accommodate some displacement away from the inner wall when the treatment catheter 210 is advanced to a position interposing the guide rail 202 and inner wall of the renal artery 12.

For example, the guide rail 202 may be delivered to the renal artery 12 in a collapsed or compressed state using a delivery sheath (see, e.g., FIG. 7) having a first diameter that is smaller than that of the renal artery 12. In this delivery configuration, the diameter of the guide rail 202 is no greater than the first diameter of the delivery sheath. Upon removal of the delivery sheath from the helical section 207, the guide rail 202 assumes its predefined helical shape having a second diameter greater than the first diameter and about equal to that of the renal artery 12. It is to be understood that a diameter considered to be “about equal to that of the renal artery” is one that provides for some degree of contact between the guide rail 202 and inner wall of the renal artery sufficient to stabilize the guide rail 202 within the renal artery.

In other embodiments, the guide rail 202 has a first diameter that is less than that of the renal artery 12 when delivered to the renal artery 12, and is forcibly increased to a second diameter about equal to a diameter of the renal artery 12 when in a deployed configuration. For example, the guide rail 202 may be delivered to the renal artery 12 in a collapsed or compressed state using a delivery sheath having a first diameter that is smaller than that of the renal artery 12. The diameter of the guide rail 202, when it this delivery configurations, is no greater than the first diameter of the delivery sheath. Upon removal of the delivery sheath from the helical section 207, the guide rail 202 assumes its predefined helical shape having a second diameter greater than the first diameter of the delivery sheath but less than that of the renal artery 12 (e.g., 10%-90% smaller). An expansion mechanism, which may be a separate apparatus or an in-situ mechanism, is employed to expand the helical section 207 of the guide rail 202 so that the helical section 207 has a third diameter greater than the second diameter and about equal to that of the renal artery 12.

The treatment catheter 210 of the denervation therapy apparatus 200 shown in FIG. 4A includes a lumen dimensioned to receive the guide rail 202. As shown in FIG. 4A, the treatment catheter 210 is configured to track over the guide 202, such as in a manner similar to various known over-the-wire catheter/lead arrangements. The treatment catheter 210 includes a treatment element 212, preferably situated at or near a distal tip 214 of the treatment catheter 210. The distal tip 214 is preferably configured as an atraumatic tip that minimizes trauma to vessel walls.

The treatment catheter 210 may be configured to delivery denervation therapy to innervated renal vasculature using a variety of technologies. According to some embodiments, the treatment catheter 210 includes a fluid transport arrangement for fluidly communicating a thermal transfer agent to and from the treatment element 212 to thermally treat innervated renal vasculature. For example, the treatment element 212 may be configured to receive a cryogenic agent to freeze nerve fibers innervating the renal artery 12.

In other embodiments, the treatment element 212 includes a radiofrequency (RF) heating arrangement configured to electrically couple with an RF generator for thermally treating innervated renal vasculature with heat. The RF generator and heating arrangement may be configured to respectively generate and receive microwave energy, for example. In further embodiments, the treatment element 212 includes a laser arrangement configured to treat innervated renal vasculature with energy emitted from a laser source.

According to other embodiments, the treatment element 212 may be configured to deliver a pharmacological agent or mixture of agents (e.g., a neurotoxin or venom) to the renal artery. In some embodiments, the treatment element 212 may be configured to deliver brachytherapy to innervated renal vasculature, such as by exposing the renal artery to radioactive material or seeds (e.g., iodine-125 or palladium-103 for low dosage rate brachytherapy, iridium-192 for high dose rate brachytherapy).

The treatment element 212 is coupled to a treatment source and serves to supply a treatment agent to the treatment element 212. The treatment source may be external to the body, implantable (temporarily or chronically), or comprise external and implantable elements. In some embodiments, the treatment source is physically connected to the treatment element 212, and the agent is communicated to the treatment element 212 via the connection. In other embodiments, the treatment source is physically separate from the treatment element 212, and the agent is communicated or coupled to the treatment element 212 by means other than a physical connection with the treatment element 212. In further embodiments, different agents and means for communicating or coupling the agent to the treatment element 212 may be employed.

It can be appreciated that the type of agent will vary in accordance with the particulars of the treatment source and treatment element 212, examples of which include a thermal transfer fluid (hot or cold), a pharmacological agent(s), radioactive material or seeds, or electromagnetic energy (e.g., RF, microwave, laser/light, ultrasonic). In some embodiments, a combination of denervation therapy apparatuses of disparate type or technology can be used together (concurrently or sequentially) to enhance the efficacy of renal denervation therapy. Combinations of disparate denervation therapy apparatuses may provided for improved therapy outcomes with reduced tissue trauma when compared to renal denervation approaches that employ one type of denervation therapy apparatus.

Details of these and other denervation therapy apparatuses and methods are described hereinbelow and in commonly owned U.S. patent application No. 13/086,121; U.S. patent application No. 13/086,116; and U.S. patent application Ser. No. 12/980,952, each of which is incorporated herein by reference.

In some embodiments, renal denervation therapy is initiated with the distal tip 214 of the treatment catheter 210 positioned at or near a distal portion 205 of the helical section 207. With the guide rail 202 remaining relatively stationary, the treatment catheter 210 is retracted in a proximal direction, allowing the treatment element 212 to deliver denervation therapy to the renal artery 12 while traveling on a helical path dictated by the helical section 207 of the guide rail 202. The treatment catheter 210 may be longitudinally displaced in a continuous motion or in a step-wise fashion.

In other embodiments, renal denervation therapy is initiated with the distal tip 214 of the treatment catheter 210 positioned at or near a proximal portion 206 of the helical section 207. With the guide rail 202 remaining relatively stationary, the treatment catheter 210 is advanced in a distal direction, allowing the treatment element 212 to deliver denervation therapy to the renal artery while traveling on a helical path dictated by the helical section 207 of the guide rail 202. The treatment catheter 210 may be longitudinally displaced in a continuous motion or in a step-wise fashion. In other embodiments, the treatment catheter 210 may be advanced by a physician in proximal and distal directions during a renal denervation procedure as desired.

One or more sensors may be employed to measure one or parameters (e.g., temperature, impedance) useful for determining the efficacy and/or extent of denervation therapy delivered to the renal artery 12. Such sensors may be incorporated as part of the denervation therapy apparatus 200 or a separate apparatus (which may be an intravascular or extravascular apparatus). Sensor measurements taken during denervation therapy can provide useful feedback to the physician. The rate of treatment element travel along the spiral section 207 of the guide rail 202 may be moderated by the physician in response to real-time sensor information.

It is noted that, in the embodiment illustrated in FIG. 4A (and other embodiments), the treatment catheter 210 need not be rotated in order to fully treat desired regions of the renal artery 12, which can reduce the risk of injuring access vasculature and aortal/renal vasculature that contacts the treatment catheter 210. Because rotation of the treatment catheter 210 is effectively accomplished by the catheter 210 tracking over the helical section 207 of the guide rail 202, the design of the treatment catheter 210 may be significantly simplified, such as by reducing or eliminating a braid arrangement or other torque-strengthening enhancements.

FIG. 4B illustrates portions of a denervation therapy apparatus 200 configured for deployment within a renal artery 12 of a patient in accordance with other embodiments of the present invention. The embodiment shown in FIG. 4B is similar in most regards to that illustrated in FIG. 4A, but differs primarily in terms of the construction of the treatment catheter 210. As shown in FIG. 4B, the distal end of the treatment catheter 210 includes a multiplicity of treatment elements 212a-212n. The treatment elements 212a-212n are preferably spaced apart from one another and arranged so that the treatment elements 212a-212n collectively complete at least one revolution of the therapy delivery portion of the treatment catheter's distal end when positioned at the helical section 207 of the guide rail 202.

In use, the treatment catheter 210 is preferably advanced over the helical section 207, and denervation therapy is delivered to the renal artery 12 in accordance with a “one-shot” treatment approach. The term “one-shot” treatment refers to treating the entirety of a desired portion of innervated vascular tissue, such as the renal artery 12, without having to move the treatment arrangement 212 to other vessel locations in order to complete the treatment procedure (as is the case for a step-and-repeat denervation therapy approach).

A one-shot treatment approach according to the embodiment shown in FIG. 4B advantageously facilitates delivery of denervation therapy that treats at least one location of each nerve fiber passing through the renal artery 12 without having to reposition the treatment catheter's distal end during denervation therapy. Embodiments of the present invention allow a physician to position the therapy delivery portion of the treatment catheter's distal end at the helical section 207 of the guide rail 202, and completely treat innervated tissue of the renal artery 12 without having to move the treatment elements 212a-212n to new vessel locations.

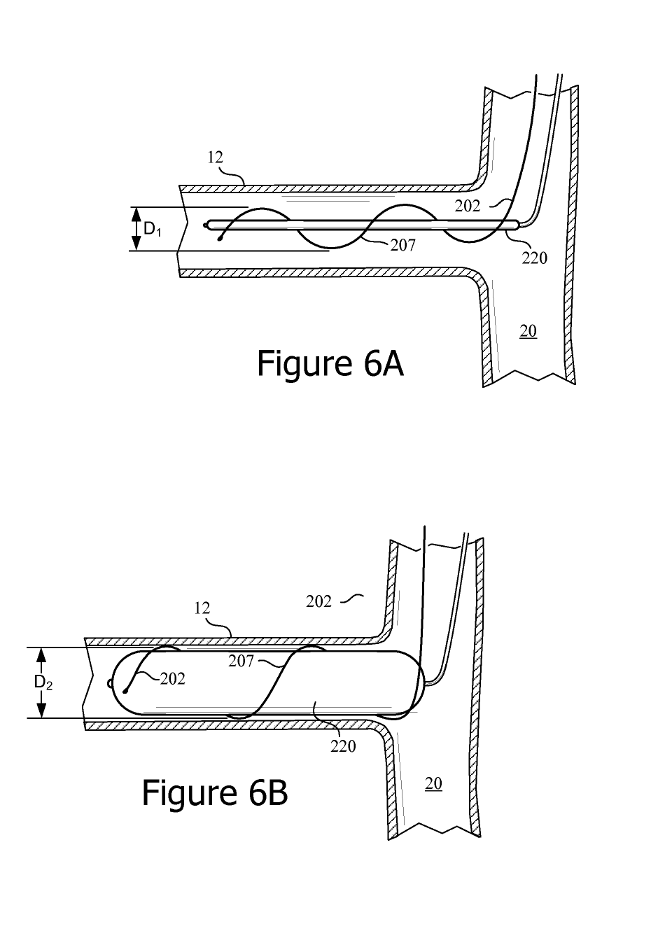

FIGS. 6A and 6B illustrate portions of a denervation therapy apparatus 200 configured for deployment within a renal artery 12 of a patient in accordance with embodiments of the present invention. According to this embodiment, a guide rail 202 includes a helical section 207 which is deformable in response to a biasing force, such as that provided by a balloon arrangement 220. The helical section 207 preferably comprises a material that is deformable and shape-retentive after being deformed.

According to the embodiment shown in FIGS. 6A and 6B, a guide rail 202 includes a helical section 207 that has an initial diameter, D₁, that is smaller than an inner diameter of the renal artery 12. Preferably, the initial diameter, D₁, of the helical section 207 relative to the diameter of the renal artery 12 is insufficient to allow the helical section 207 to contact the inner wall of the renal artery 12 if suspended with the lumen of the renal artery 12, as is depicted in FIG. 6A. After positioning the helical section 207 of the guide rail 220 and an uninflated balloon 220 within the lumen of the renal artery 12, the balloon 220 is inflated to contact the helical section 207 of the guide rail 202. The balloon 220 is further pressurized, which produces an outwardly directed biasing force that causes the helical section 207 to expand and achieve a desired second diameter, D₂, which is depicted in FIG. 6B. The second diameter, D₂, is preferably about the same diameter as the renal artery 12. The balloon 220 is deflated and removed from the patient. The helical section 207 retains is expanded shape, with a diameter substantially the same as the second diameter, D₂.

The balloon 220 may be delivered to the renal artery 12 with the balloon 220 pre-positioned within the helical section 207. Alternatively, the balloon 220 may be advanced into the helical section 207 after initially positioning the helical section 207 in the lumen of the renal artery 12. The guide rail 202 and balloon 220 are typically delivered to the renal artery 12 with the aid of a delivery sheath, such as a guide catheter. FIG. 7 shows a cross-section of components of a denervation therapy apparatus 200 according to the embodiment illustrated in FIGS. 6A and 6B, which includes a delivery sheath 219 (e.g., guide catheter), helical section 207 of a guide rail 202, and a balloon 220, all of which are encompassed by an inner wall of a renal artery 12.

The helical section 207 preferably comprises a material that is deformable and shape-retentive after being deformed. For example, the helical section 207 may comprise a material or composite that is plastically deformable, such that the helical section 207 retains its expanded shape upon removal of a force that causes deformation. The balloon 220 may a compliant or semi-compliant balloon having a conventional construction.

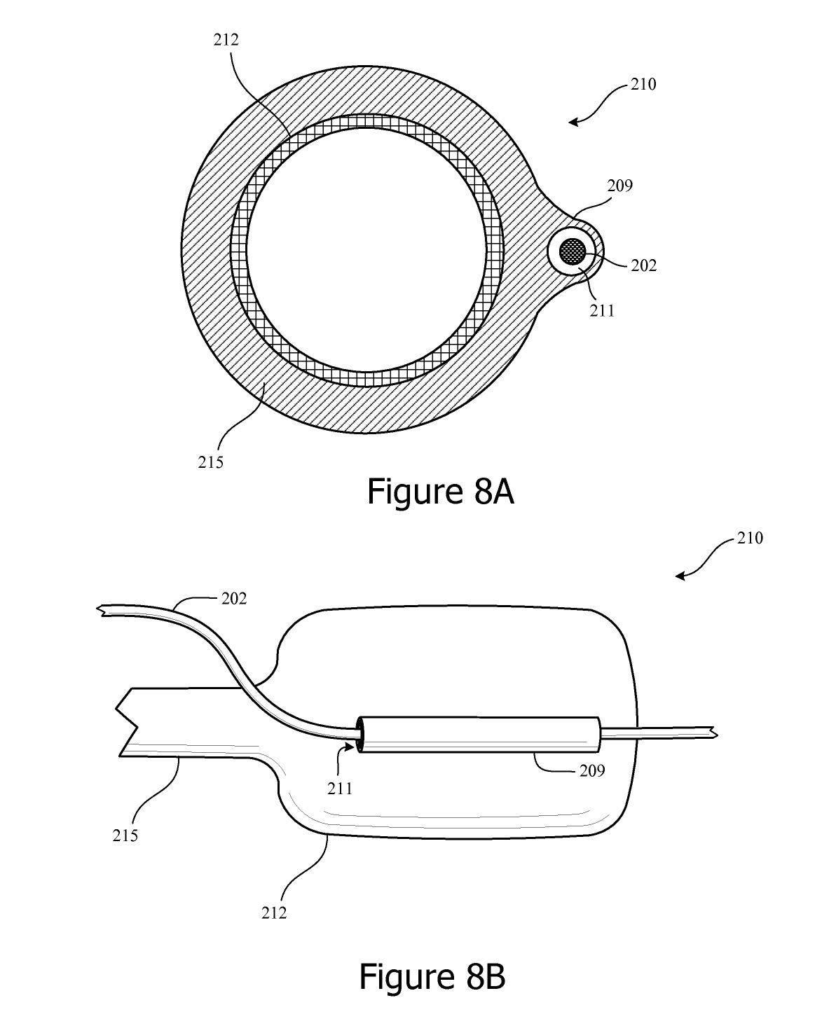

FIGS. 8A and 8B illustrate a treatment catheter 210 implemented in accordance with other embodiments of the present invention. FIG. 8A is a cross-section of a treatment catheter 210 that shows a sidewall 209 extending from an outer surface of a sheath 215 of the treatment catheter 210. The cross-section also shows a representative treatment element 212 (e.g., an RF heating element or cryotherapy element). The sidewall 209 includes a lumen 211 having a diameter dimensioned to receive a guide rail 202 of a type previously described. In the embodiment shown in FIG. 8A, the sidewall 209 extends along the length of the treatment catheter 210, from a proximal end of the treatment catheter 210 to a distal end of the treatment catheter 210. It is noted that the lumen 211 may be formed in the wall of the sheath 215, allowing the sheath 215 to maintain a substantially cylindrical shape along its length.

In one configuration, the diameter of the sheath 215 is fairly uniform (or changes fairly uniformly) along its length. This diameter may increase somewhat the region of the treatment element 212 situated at the distal end of the sheath 215 in order to accommodate components of the treatment element 212. In another configuration, the diameter of the sheath 215 proximal of the treatment element 212 is smaller than that at the treatment element 212.

FIG. 8B shows another treatment catheter 210 in accordance with embodiments of the present invention. FIG. 8B includes a sidewall 209 of the type described with reference to FIG. 8A, but with the sidewall 209 provided only along a distal portion of the sheath 215 of the treatment catheter 210. The sidewall 209 includes a lumen 211 having a diameter dimensioned to receive a guide rail 202 of a type previously described. In the embodiment shown in FIG. 8B, the sidewall 209 extends along a length of the treatment element 212, it being understood that the sidewall 209 may further extend along a short length of the sheath 215 proximate the treatment element 212.

According to one approach, the guide rail 202 is first delivered into the lumen of the renal artery 12, which may involve use of a delivery sheath 219 (e.g., guide catheter). With the guide rail 202 in its deployed configuration within the renal artery 12, the treatment catheter 210 is threaded onto the guide rail 202 by insertion of the guide rail's proximal end into the lumen of the sidewall 209. Tracking along the guide rail 202, the treatment catheter 210 is advanced through access vasculature and into the lumen of the renal artery 12. A delivery sheath 219 may be used to facilitate advancement of the treatment catheter 210 into the renal artery 12. Alternatively, the treatment catheter 210 may be advanced into the renal artery 12 without use of the delivery sheath 219, such as by tracking along the guide rail 202 in a manner similar to an over-the-wire deployment approach.

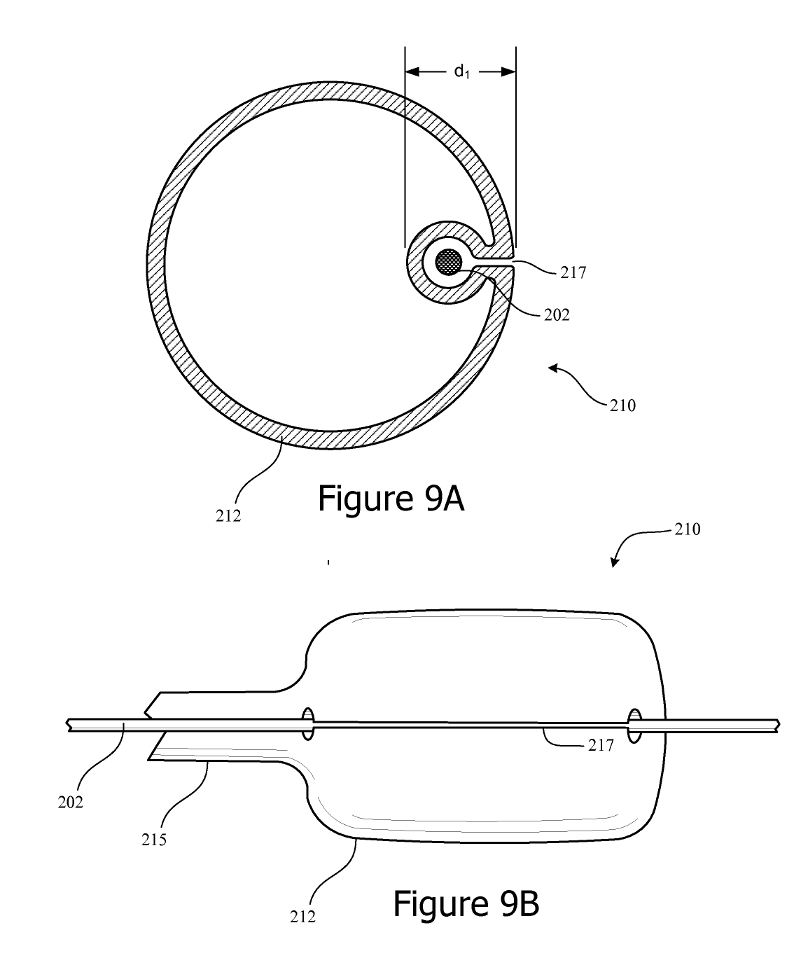

FIGS. 9A and 9B illustrate a treatment catheter 210 implemented in accordance with further embodiments of the present invention. FIG. 9A is a cross-section of a treatment catheter 210 that includes a channel 217 formed along the length of the sheath 215 of the treatment catheter 210. The channel 217 has a shape configured to receive and capture a guide rail 202 of a type previously described. The channel 217 is shown to have a depth, d₁, which can be selected to situate the channel 217 at a desired distance relative to the outer surface of the sheath 215 (or relative to the central axis of the sheath 215). In some embodiments, the channel 217 and guide rail 202 may be shaped so that rotation of the treatment element 212 is prevented as the treatment element 212 tracks along the guide rail 202 (e.g., a longitudinal “T” shaped channel). Prevention of treatment element rotation can provide predictable positioning of the treatment element 212 relative to the inner wall of the renal artery 12. In other embodiments, the channel 217 and guide rail 202 may be shaped to allow for rotation of the treatment element 212.

FIG. 9B shows another treatment catheter 210 in accordance with embodiments of the present invention. The embodiment shown in FIG. 9B is similar to that illustrated in FIG. 9A, but includes a channel 217 formed only along a length of the distal portion of the sheath 215. In FIG. 9B, the channel 217 extends along the length of the treatment element 212, it being understood that the channel 217 may further extend along a short length of the sheath 215 proximate the treatment element 212.

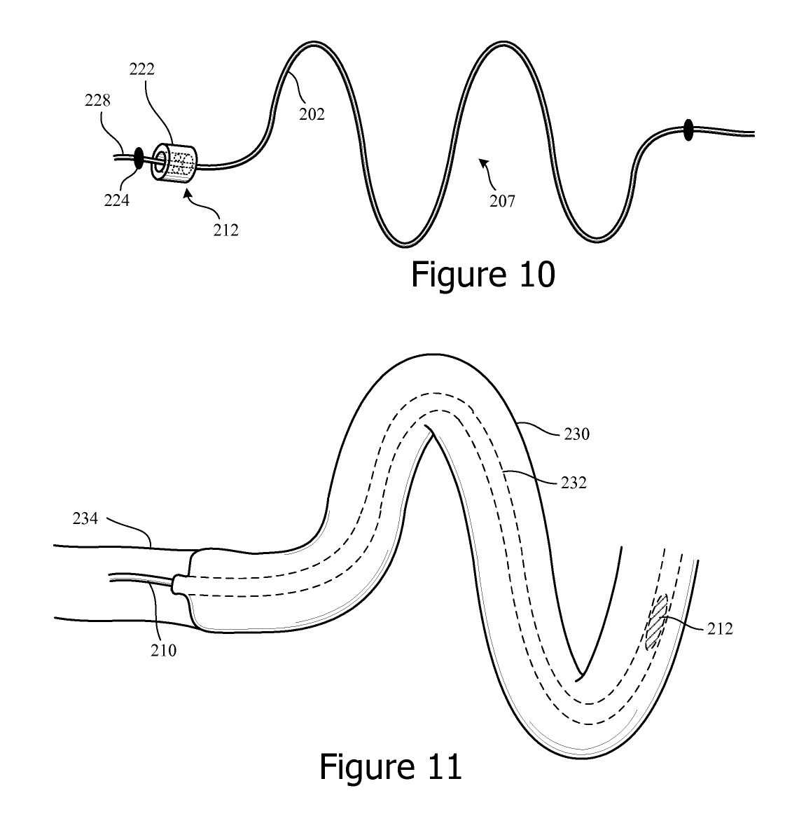

FIG. 10 illustrates a treatment element 212 in accordance with other embodiments of the present invention. The embodiment shown in FIG. 10 is particularly useful for treatment element configurations that do not require a catheter or other structure to supply a treatment agent to the treatment element 202 via access vasculature. Such treatment element configurations include those that incorporate electromagnetic (e.g., inductive) or radioactive treatment elements 212, for example.

In FIG. 10, the treatment element 212 comprises a carriage member 222 which is configured to travel along at least the helical section 207 of the guide rail 202. The carriage member 222 may comprises a flexible tube member that can bend as the carriage member 222 is advanced along the helical section 207 of the guide rail 202. Denervation therapy components of the treatment element 212 are preferably mounted to the carriage member 222. A push wire 228 may be configured to detachably couple with the carriage member 222 and used to move the carriage member 222 of the treatment element 212 along the helical section 207.

In some configurations, a proximal stop 224 and a distal stop 226 are respectively positioned at proximal and distal locations of the helical section 207. The proximal and distal stops 224 and 226 limit the longitudinal travel of the carriage member 222 and the treatment element 212 to the region of the guide rail 202 that includes the helical section 207. In other embodiments, the guide rail 207 includes only a distal stop 226, allowing the carriage member 222 and treatment element 212 to travel from the proximal end of the guide rail 202, along the helical section 207, and to the distal stop 226.

FIG. 11 shows a treatment element 212 of a treatment catheter 210 in accordance with further embodiments of the present invention. In the embodiment shown in FIG. 11, the treatment element 212 includes a balloon 230 having a generally helical shape. The balloon 230 is provided on a distal end of a shaft 234, which may have an inflation lumen provided therethrough. The distal end of the shaft 234 may have a flexible pre-shaped section that is collapsible when placed in the lumen of a delivery sheath, and expands to assume a helical shape upon removal of the delivery sheath. Alternatively, the balloon 230 may have a lumen dimensioned to receive a shaping member received from a lumen of the shaft 234 that contorts the balloon 230 to assume a helical shape when positioned within the balloon's lumen. The balloon 230 may be constructed as a compliant, semi-compliant, or non-compliant balloon depending on design and implementation particulars.

In some embodiments, the balloon 230 includes a channel 232 provided along the spiral therapy delivery portion of the balloon 230. The channel 232 may be provided in or on the balloon 230 in a manner previously described. For example, the channel 232 may define a lumen or hollow sidewall of the balloon 230, a channel recessed in the wall of the balloon 230/shaft 234, or a channel disposed on the outer surface of the balloon 230. A treatment element 212 is preferably configured to track through, on, over or along the channel 232 in a generally spiral pattern.

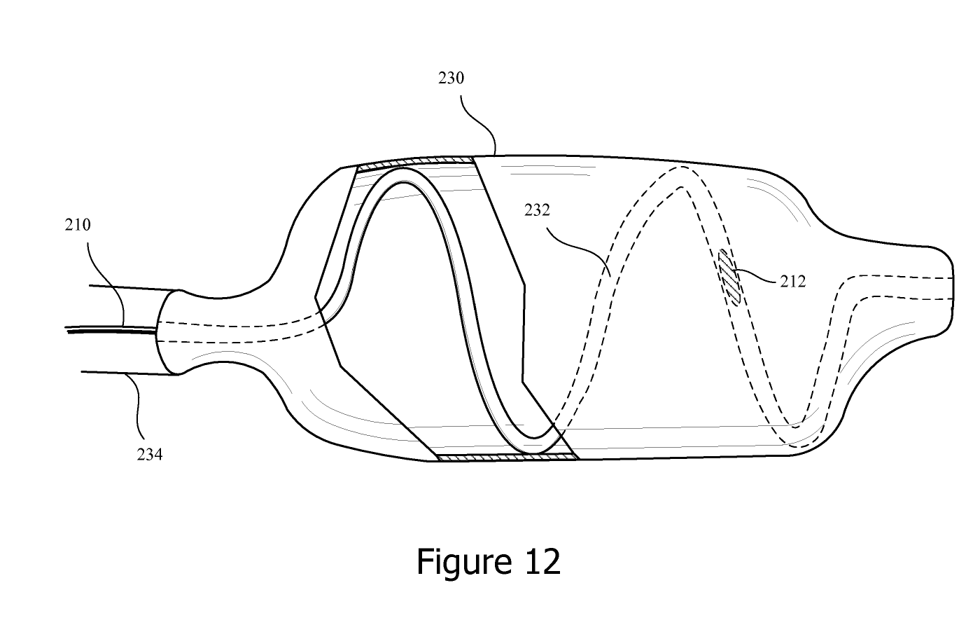

FIG. 12 illustrates a further embodiment of a treatment element 212 of a treatment catheter 210 in accordance with the present invention. In FIG. 12, the treatment element 212 includes a balloon 230 having a generally cylindrical shape. The balloon 230 is provided on a distal end of a shaft 234, which may have an inflation lumen provided therethrough. The balloon 230 includes a longitudinal channel 232 having a generally spiral shape provided along the therapy delivery portion of the balloon 230. The spiral channel 232 may be provided in or on the balloon 230 in a manner previously described. For example, the spiral channel 232 may define a lumen or hollow sidewall of the balloon 230, a channel recessed in the wall of the balloon 230/shaft 234, or a channel disposed on the outer surface of the balloon 230. A treatment element 212 is preferably configured to track through, on, over or along the channel 232 in a generally spiral pattern. The balloon 230 may be constructed as a compliant, semi-compliant, or non-compliant balloon depending on design and implementation particulars.

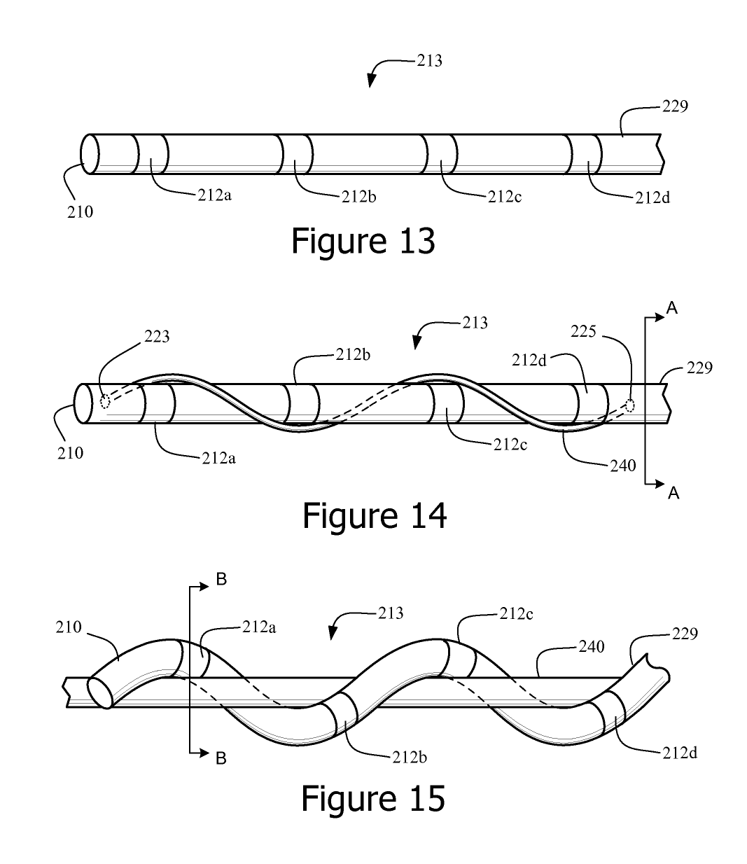

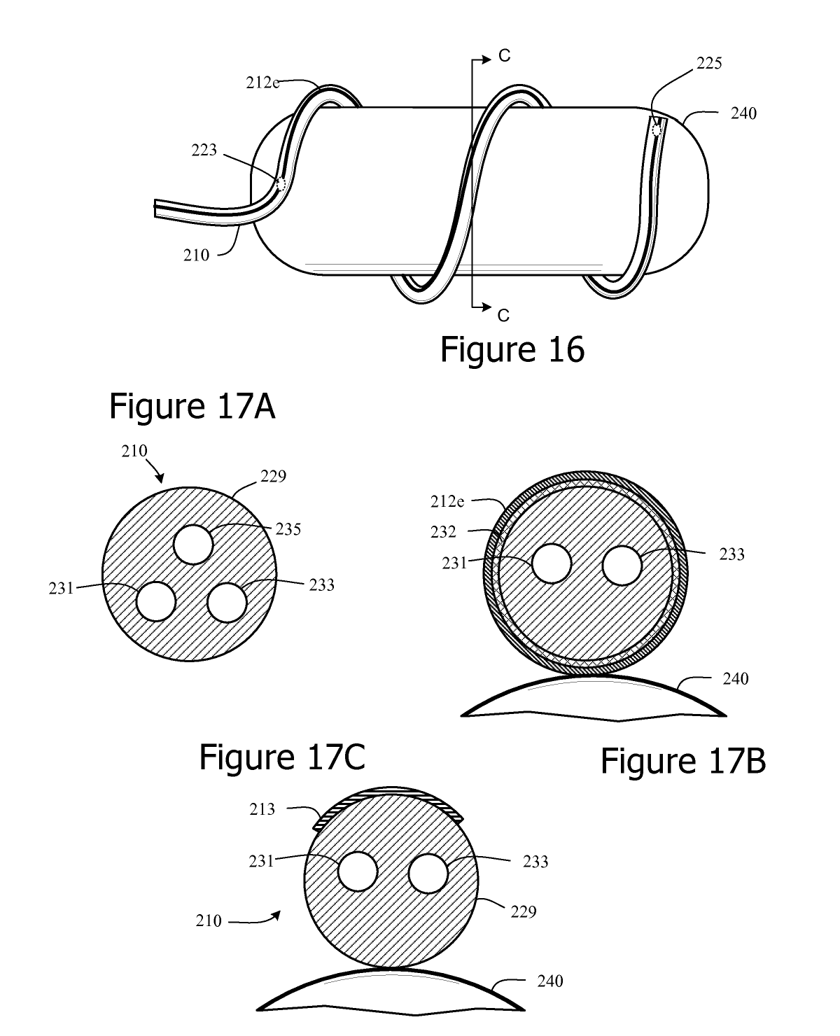

FIGS. 13-16 illustrate a denervation therapy apparatus configured for deployment within a renal artery of a patient in accordance with embodiments of the present invention. The denervation therapy apparatus shown in FIGS. 13-16 includes a treatment catheter 210 and a balloon arrangement 230. In FIGS. 13-15, the treatment catheter 210 includes a multiplicity of spaced-apart treatment elements provided at the distal end of the catheter 210. As shown, the treatment catheter 210 includes four treatment elements, 212a-212d, it being understood that more or fewer than four treatment elements may be employed. In FIG. 16, the treatment catheter 210 includes a continuous longitudinally extending treatment element 212 situated along a length of the catheter's distal end.

The distal end of the treatment catheter 210 that encompasses the treatment section 213 shown in FIGS. 13-16 is formed of a relatively flexible material, which allows for multi-planar flexing of the treatment section 213. A balloon 240 is arranged at the distal end of the treatment catheter 210 such that it forms a spiral of at least one turn along the treatment section 213 of the treatment catheter 210. In one configuration, the balloon 240 is loosely wrapped around the treatment section 213 of the treatment catheter 210 in a spiral pattern. The balloon 240 shown in FIG. 14 includes a distal tether 223 that connects the distal end of the balloon 240 to a distal end of the treatment section 213. The balloon 240 is also shown to include a proximal tether 225 that connects the proximal end of the balloon 240 to a proximal end of the treatment section 213.

Tethering the balloon 240 to the distal end of the treatment catheter 210 at two or more tether locations allows the balloon to shift somewhat as it expands from its non-inflated configuration (shown FIG. 14) to its inflated configuration (shown in FIGS. 15 and 16). It is understood that other attachment arrangements may be employed to connect the balloon 240 to the distal end of the treatment catheter 210. For example, a continuous or discontinuous seam having a spiral shape may be formed between the balloon and the distal end of the treatment catheter 210.

As is best shown in FIGS. 15 and 16, inflation of the balloon 240 causes the balloon 240 to stiffen and assume a substantially elongated cylindrical shape. The balloon 240 straightens during inflation, resulting in tensioning of the distal and proximal tethers 223, 225, which causes the relatively flexible treatment section 213 at the distal end of the treatment catheter 210 to contort into a substantially spiral shape. The materials and dimensions of the treatment section 213 and the balloon 240 are preferably selected to allow the treatment section 213, with the balloon 240 inflated, to assume a spiral that has a diameter sufficient to facilitate contact between at least portions of the treatment section 213 and the inner wall of the renal artery 12. For example, the balloon 240 may have a compliant or semi-compliant balloon construction. The length of the balloon 240 may range from about 2 cm to about 5 cm. The diameter of the balloon 240, when inflated, may range from about 5 mm to about 10 mm.

In the embodiment shown in FIG. 15, forcing the distal end of the treatment catheter 210 to assume a substantially helical shape using the balloon 240 urges the four spaced-apart treatment elements, 212a-212d, of the treatment section 213 into contact with four regions of the renal artery's inner wall. The four treatment elements, 212a-212d, have a size (longitudinally and/or circumferentially) and spacing (preferably approximately equally spaced) relative to one another such that the four treatment elements, 212a-212d, contact the inner renal artery wall at 0°, 90°, 180°, and 270° locations about the renal artery 12. It can be appreciated that the spaced-apart treatment elements, 212a-212d, of the treatment section 213, when urged into contact with four regions of the renal artery's inner wall by the balloon 240, are advantageously positioned to ensure that each nerve fiber passing along the renal artery wall is subject to denervation therapy.

FIG. 16 illustrates an embodiment which is a variation of that shown in FIGS. 13-15. In FIG. 16, the treatment section 213 of the treatment catheter includes a continuous treatment element 212e of a predefined length and width. The length and width of the continuous treatment element 212e are preferably selected to ensure that, when urged into contact with the inner renal artery wall upon inflation of the balloon 240, the contacting portions of the treatment element 212e collectively complete as least one 360° turn of the renal artery 12. In some configurations, a single continuous treatment element 212e of predetermined length and width is disposed axially along the distal end of the treatment catheter 210. In other configurations, two or more continuous treatment elements 212e of predetermined length and width are disposed axially along the distal end of the treatment catheter 210 in a circumferentially spaced-apart fashion.

In accordance with another embodiment of the invention depicted in FIG. 16, the continuous treatment element 212e comprises a long continuous conductor which contacts the wall as it spirals along the inner renal artery wall. Preferably, a ribbon electrode 212e is wound around the distal end of the treatment catheter 210 in a barber pole configuration with little or not space between successive turns to form a single electrode with a plurality of electrodes provided thereon. The plurality of electrodes may be connected electrically with an insulating coating applied periodically to make independent burns. In another configuration, each electrode may have its own independent electrical wire.

After the balloon 240 is inflated, the spiral ribbon electrode 212e touches the wall of the renal artery 12 in spots relatively close together. Ablation using the entire ribbon electrode 212e with a monopolar mode to a return back pad, for example, can create a spiral of spots along the renal artery wall. The treatment can be continued for a duration sufficient to make the spots merge into a continuous spiral, or left as a series of spots of adequate depth. The benefit of this approach is a short treatment time, since only one RF application is required (e.g., a one-shot procedure). A temperature sensor(s) can be incorporated into one or more locations in the spiral electrode 212e.

In the embodiments illustrated in FIGS. 13-16, the treatment catheter 210 may be configured to delivery denervation therapy to innervated renal vasculature using a variety of technologies. In various embodiments, the treatment element 212 comprises one or more electrodes (e.g., electrodes 212, 212a-212d, 212e), and the treatment catheter 210 is configured to deliver RF ablation therapy to the renal artery 12. The RF ablation catheter 210 is preferably configured to have a monopolar configuration, with each electrode 212, 212a-212d, 212e at the treatment section 213 electrically coupling with a return back pad or other patient-external return electrode.

Each electrode site may be treated separately (e.g., sequentially) or all sites can be treated concurrently. A temperature sensor is preferably included on the inner wall of each electrode band, such as for electrodes 212, 212a-212d. For a continuous electrode, such as electrode 212e, multiple temperature sensors may be included at different locations along the inner wall of the continuous electrode. An RF generator (e.g., a patient external system) electrically couples to each of the electrodes and the back electrode, with RF power driven to achieve a target temperature for a specified time in order to create the desired size of lesion in the renal artery wall. Using temperature as a feedback parameter, the lesion depth can be controlled and steam pops avoided.

FIGS. 17A-17C are cross-sections of a distal portion of the treatment catheter apparatus 200 in accordance with embodiments of the invention. FIG. 17A is a cross-section of the treatment catheter 210 shown in FIG. 14 taken along section A-A proximal of the treatment section 213. FIG. 17B is a cross-section of the treatment section 213 of the treatment catheter's distal portion shown in FIG. 15 taken along section B-B. FIG. 17C is a cross-section of the treatment section 213 of the treatment catheter's distal portion shown in FIG. 16 taken along section C-C. It is noted that the electrode 213 may extend 360° around the shaft 229 if desired, as is shown in FIG. 17B.

FIG. 17A shows a shaft 229 of the treatment catheter's distal end, which includes a multiplicity of lumens. The lumens include an inflation lumen 235 which is fluidly coupled to the balloon 240 and a patient-external fluid source. A pressurized fluid (e.g., saline and x-ray contrast) is injected into, and extracted from, the inflation lumen 235 to respectively inflate and deflate the balloon 240. A second lumen 231 is preferably configured to receive one or more conductors for electrically coupling to one or more electrodes 212, 212a-212d, 212e. If two or more conductors are disposed within the second lumen 231, these conductors are covered with electrical insulation or can be disposed within separate lumens. A third lumen 233 may be provided for other uses, such as for receiving a guide wire to facilitate over-the-wire deployment of the treatment catheter 210 into the renal artery 12. The third lumen 233 and other lumens may be provided for various purposes, including for receiving a temperature sensor, a visualization arrangement, a shaping or guiding stylet, or a pharmacological agent, for example.

Preferably, the inflation lumen 235 is disposed within the shaft 229 of the treatment catheter 210 and extends from a proximal end of the catheter 210 to a location proximate the treatment section 213. At the treatment section 213, the inflation lumen 235 extends to an outer surface of the shaft 229, and is fluidly coupled to the proximal end of the balloon 240, defining an inlet of the balloon 240. In other configurations, the inflation lumen 235 may extend along at least a portion of an exterior wall of the shaft 229.

FIG. 17B shows a balloon 240 (inflated) having an outer wall that is in contact with an outer wall of a shaft 229 of the treatment catheter's distal end. The cross-section of FIG. 17B shows an annular or band electrode 212a disposed circumferentially about the shaft 229 and the second and third lumens 231 and 233 described above. It is noted that the cross-section of FIG. 17B does not show the inflation lumen 235, since this lumen 235 terminates at the outer surface of the shaft 229 near the proximal end of the treatment section 213. The cross-section of FIG. 17C shows a portion of a ribbon electrode 212e shown in FIG. 16 disposed about a portion of the shaft's circumference. As in the case of FIG. 17B, the cross-section of FIG. 17B includes the second and third lumens 231 and 233 described above, but does not show the inflation lumen 235, since this lumen 235 terminates at the outer surface of the shaft 229 near the proximal end of the treatment section 213.

Although described above in the context of RF ablation, other denervation technologies may be used in the embodiments shown in FIGS. 13-16. For example, the RF generator and electrode arrangement provided at the distal end of the treatment catheter 210 may be configured to respectively generate and receive microwave energy. In further embodiments, the treatment section 213 of the treatment catheter 210 may include a laser arrangement configured to treat innervated renal vasculature with energy emitted from a laser source. In some embodiments, the treatment section 213 of the treatment catheter 210 may include an ultrasonic arrangement configured to treat innervated renal vasculature with ultrasound emitted from an ultrasound source.

In other embodiments, the treatment section 213 includes a fluid transport arrangement for fluidly communicating a thermal transfer agent to and from the treatment section 213 (e.g., via elements 212a-212d or continuous element 212e) to thermally treat innervated renal vasculature using a heated fluid or a cryogenic agent. In such embodiments, the shaft 229 includes appropriate supply and return lumens to facilitate circulation of the thermal transfer fluid and gas to and from the treatment section 213 of the catheter 210.

In alternative embodiments, the treatment section 213 may be configured to deliver a pharmacological agent or mixture of agents (e.g., a neurotoxin or venom) to the renal artery. In some embodiments, the treatment section 213 may be configured to deliver brachytherapy to innervated renal vasculature. These and other therapy technologies can be employed using a treatment catheter 210 suitable for a given therapy technology in combination with a spiral shape-forcing balloon 240 in accordance with the present invention. Details of these and other denervation therapy apparatuses and methods are described herein and in the documents incorporated herein by reference.

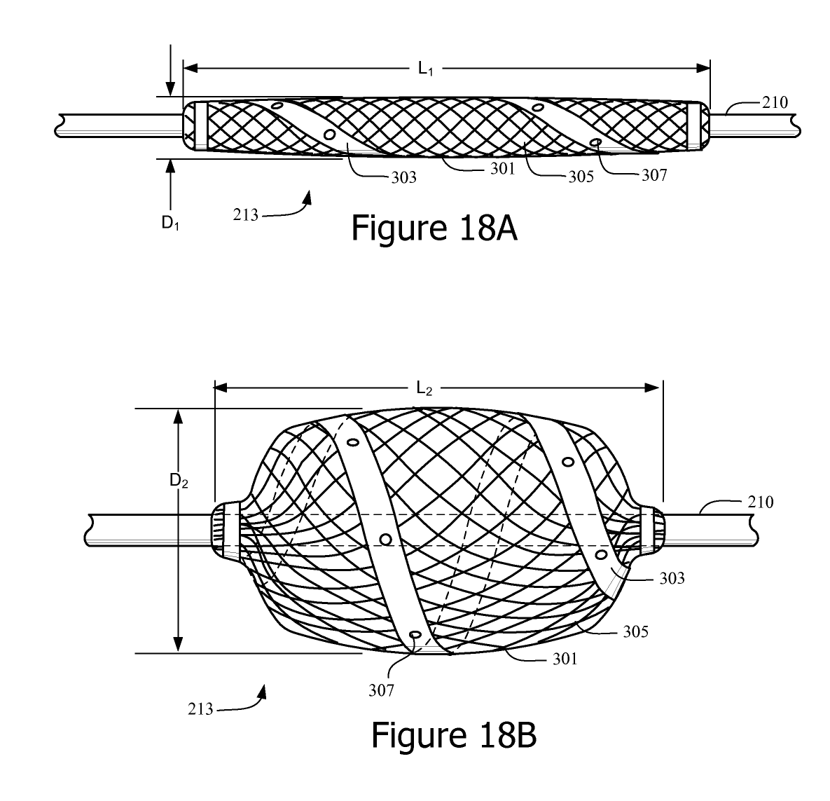

Turning now to FIGS. 18A and 18B, there is illustrated an embodiment of a treatment section 213 provided at a distal end of a treatment catheter 210 that incorporates a braid member 301 comprising an electrically conductive pattern 303 and configured to deform in the manner of a so-called Chinese handcuff. The treatment section 213, including the braid member 301 when in a relaxed state, is dimensioned for deployment within the renal artery. The braid member 301 preferably comprises a woven material having a resiliency sufficient to facilitate deployment of the braid member 301 into the renal artery from the abdominal aorta.

The braid member 301 is configured to decrease in length and increase in diameter in response to axial compression, and to increase in length and decrease in diameter in response to axial tensioning or relaxation. With no axial compression applied (e.g., when in a relaxed state), the diameter of the braid member 301 is relatively small and can readily be advanced into the renal artery. With axial compression applied, the braid member 301 shortens and the diameter increases to at least that of the renal artery, thereby urging the electrically conductive pattern 303 into contact with or close proximity of the renal artery's inner wall.

For example, the braid member 301 is shown in FIG. 18A to have a substantially cylindrical shape with a length, L₁, and a diameter, D₁ when in a relaxed or an in-tension configuration (i.e., a non-compressed state). In FIG. 18B, the braid member 301 is shown in a compressed configuration, and assumes a bulbous shape with a length, L₂, and a diameter, D₂, where D₂>>D₁ and L₂<<L₁. According to various embodiments, the diameter D₁ of the braid member 301 in a relaxed state may be about 1 mm to about 2 mm. Assuming that the renal artery has a diameter between about 5 mm and 8 mm, D₂ is typically between about 250% to about 800% greater than D₁. The braid member 301 is preferably configured to selectably assume bulbous and cylindrical shapes in response to application and removal of an axially directed compression force.

The pattern 303 preferably comprises an electrically conductive pattern having a substantially helical shape that completes at least one revolution of the braid member 301. The electrically conductive pattern 303 is configured to electrically couple with a radiofrequency generator. In some embodiments, the braid member 301 comprises filaments that are woven together in a crossed alternating configuration to form a Chinese handcuff design.

The material of the braid member 301 preferably comprises an electrically insulating material, such as a polymeric material. The braid member 301 includes insulating portions 305 defined by regions of the braid member 301 devoid of the electrically conductive pattern 303. A multiplicity of temperature sensors 307 may be incorporated at different locations within the pattern 303. Preferably, each of the temperature sensors 307 is individually addressable to provide the temperature at each temperature sensor location. Suitable temperature sensors include thermocouples and thermistors, for example.

According to some embodiments, most of the filaments of the braid member 301 are electrically nonconductive, but some filaments are conductors which are masked so that regions of the braid member 301 are conductive. These masked conductive regions preferably define a pattern 303 of electrodes 307 with a coating to insulate the ribbon between them, or it may be one continuous electrode spiral. These masked regions preferably define a pattern 303 that completes as least one revolution of the braid member 301. It is noted that voids 305 may be holes between braid filaments. The voids 305 can be insulating if a balloon is disposed inside the braid. In other embodiments, it is not necessary for voids 305 to be insulating.

The electrically conductive pattern 303 may be formed in a number of ways, including by various known spray, dipping or coating techniques. According to one embodiment, the electrically conductive pattern 303 may be formed using a conductive wire or ribbon, without masking, to form one continuous spiral electrode. The continuous spiral electrode may be woven into a braid or wound around the braid. In another embodiment, a conductive ribbon with masks may be used to create a multiplicity of electrodes around the spiral, but connected together. In a further embodiment, insulating ribbon with a multiplicity of electrodes formed thereon may be used, each with separate insulated wires. This can be a flex circuit PCB (printed circuit board) with electrodes on the outer face and separate connecting wires in the inside. This structure can be wound into a braid or wound over the braid.

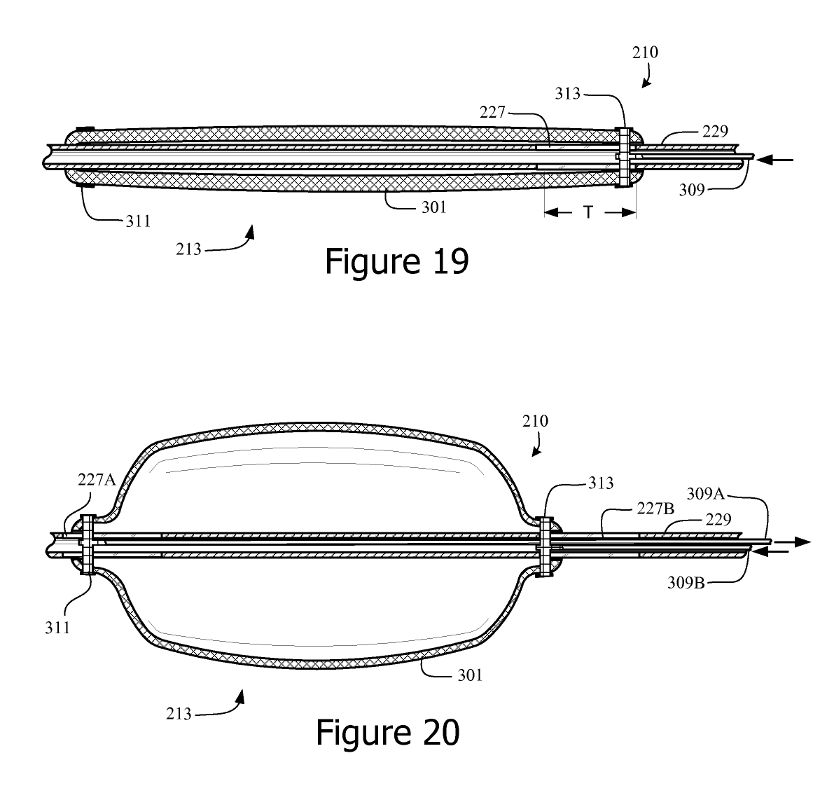

FIG. 19 illustrates an arrangement configured to actuate the braid member 301 of a treatment catheter 210 in accordance with embodiments of the invention. In FIG. 19, the distal end 311 of the braid member 301 is shown secured or otherwise held at a stationary location relative to the catheter's shaft 229. The proximal end 313 of the braid member 301 is permitted to move axially toward and away from the stationary distal end 311. An actuator 309 is coupled to the proximal end 313 of the braid member 301 and can be displaced longitudinally within a lumen of the catheter 210. In some configurations, the distal end of the actuator 309 is connected to the proximal end 313 of the braid member 301. In other configurations, a coupling arrangement is provided that facilitates releasable engagement between the distal end of the actuator 309 and the proximal end 313 of the braid member 301.

Longitudinal displacement of the actuator 309 causes the proximal end 313 of the braid member 301 to move toward or away from the stationary distal end 311 as desired. The braid member 301 can be compressed by moving the actuator 309, and therefore the proximal end 313 of the braid member 301, toward the braid member's distal end 311. Conversely, the braid member 301 can be relaxed or tensioned by moving the actuator 309, and therefore the proximal end 313 of the braid member 301, away from the braid member's distal end 311.

In the embodiment shown in FIG. 19, a slot or channel 227 is provided in the wall of the catheter's shaft 229 proximate the braid member 301. The proximal end 313 of the braid member 301 is coupled to the distal end of the actuator 309 via the slot 227. The longitudinal distance of travel, T, of the braid member's proximal end 313 is preferably limited by the axial length of the slot 227. It is understood that the configuration shown in FIG. 19 can be reversed, such that the proximal end 313 of the braid member 301 is positionally fixed, and the distal end 311 is coupled to the actuator 309 and permitted to travel axially to generate compressive and tensile forces in the braid member 301.

FIG. 20 illustrates an arrangement configured to actuate the braid member 301 of a treatment catheter 210 in accordance with other embodiments of the invention. In FIG. 20, the distal end 311 and the proximal end 313 of the braid member 301 are permitted to travel axially under control of respective actuators 309A and 309B. In this embodiment, slots 227A and 227B are provided in the wall of the catheter's shaft 229 and facilitate coupling between actuators 309A and 309B and distal and proximal ends 311 and 313 of the braid member 301, respectively. By controlling the longitudinal displacement of the actuators 309A and 309B, the distal and proximal ends 311 and 313 of the braid member 301 can be moved axially relative to one another, thereby facilitating compression, tensioning or relaxation of the braid member 301.

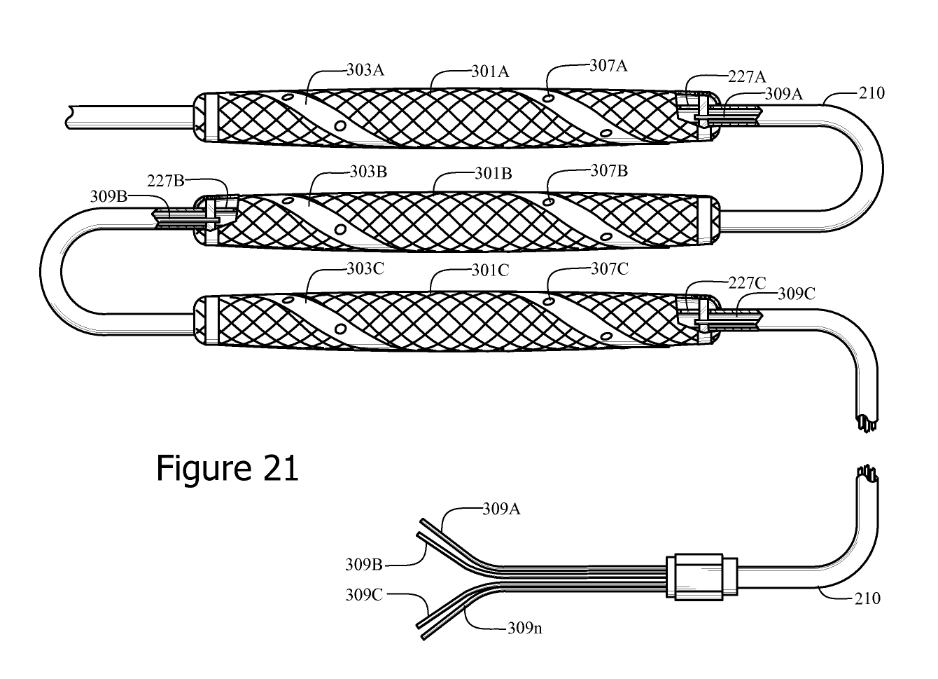

FIG. 21 shows a multiplicity of braid members 301A-301n provided at the distal end of a treatment catheter 210 in accordance with embodiments of the invention. In FIG. 21, each of the braid members 301A-301n is individually controlled by an actuator 309A-309n. Each of the braid members 301A-301n comprises a conductive pattern 303A-303n. Preferably, each of the braid members 301A-301n comprises a conductive pattern 303A-303n that defines a portion of a spiral, such that alignment of conductive pattern portions 303A-303n across all of the braid members 301A-301n results in a spiral shaped electrode configuration. Provision of a multiplicity of braid members 301A-301n provides for selective actuation of a particular braid member 301A-301n of the treatment catheter 210. Provision of multiple braid members 301A-301n also provides for enhanced control and sensor feedback for each braid member 301A-301n during RF denervation therapy.

It is understood that a single braid member 301, such as that shown in FIGS. 18A-20, may be configured to include two or more electrically isolated conductive patterns 303A-303n, each being separately controllable. For example, a switch can be incorporated into the treatment catheter 210 or a circuit proximal of the treatment catheter 210 that electrically couples an RF generator to a selected one of the two or more electrically isolated conductive patterns 303A-303n. In such a configuration, a separate temperature sensor 307 is provided for each electrically isolated conductive pattern 303A-303n.

In accordance with some denervation therapy approaches, the treatment catheter 210 is advanced to a patient's renal artery with the braid member 301 in a relaxed or tensioned state. The resiliency and small profile of the braid member 301 enhances maneuverability of the braid member 301 around the near 90 degree turn from the abdominal aorta and into the renal artery. When properly positioned within the renal artery, the braid member 301 is compressed, causing the braid member's diameter to increase so that the conductive pattern 303 comes into close proximity or contact with the renal artery's inner wall.

The conductive filaments of the braid member's pattern 303 are energized using an RF generator preferably in a monopole mode to create RF ablation lesions in the renal artery where the conductive pattern 303 is un-insulated. Preferably, the conductive filaments of the braid member 301 are fashioned so that un-insulated regions of the braid member 301 line up in a spiral pattern. This allows a spiral lesion to be created at the same time (i.e., a one-shot therapy approach), thus disrupting renal nerve function in the wall of the renal artery. This approach provides for creation of a desired spiral lesion in a minimal amount of time. After completing the denervation therapy for each of the patient's renal arteries, the compressive force on the braid member 301 is relieved, allowing the braid member 301 to assume its compact cylindrical profile. The braid member 301 and the treatment catheter 210 are then removed from the patient.

In accordance with other embodiments, the different regions of the conductive pattern 301 of the braid member 301 or multiple braid members 301 can be actuated (i.e., compressed and energized) in a sequential manner. Using this approach, lesions can be created one at a time to sequentially form a series of burn spots which collectively form a spiral along the wall of the renal artery. Although slower than a one-shot therapy approach, a sequential denervation therapy approach provides for enhanced control to adapt to local changes based on feedback from temperature and/or impedance sensing arrangements.

It is noted that the braid member 301 is preferably constructed to permit blood to perfuse through the braid member 301 during RF ablation therapy. Perfusion of blood through the braid member 301 advantageously provides cooling to the inner wall of the renal artery during RF ablation therapy, thereby reducing injury to non-targeted renal artery tissue.

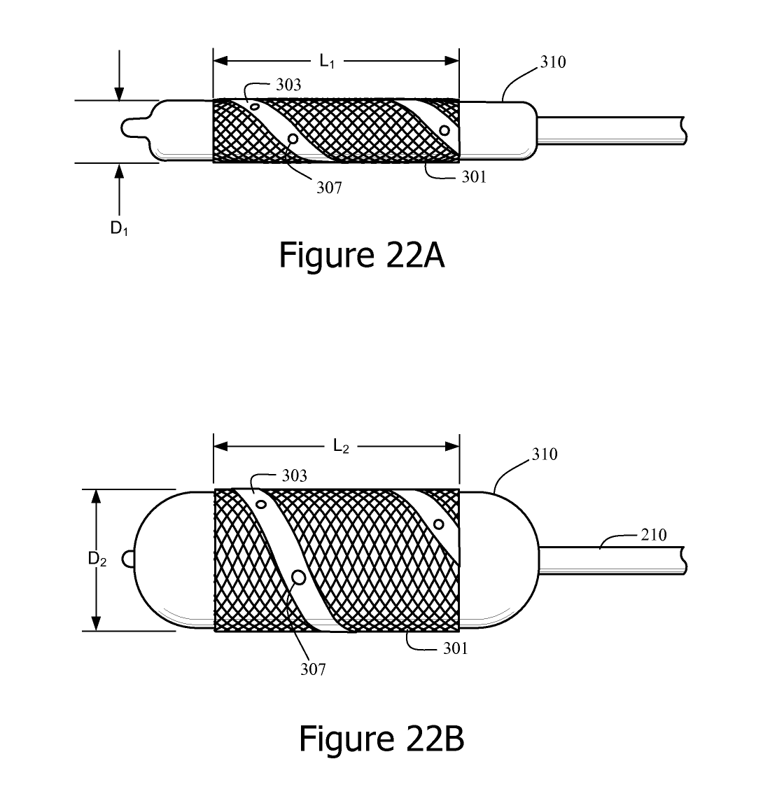

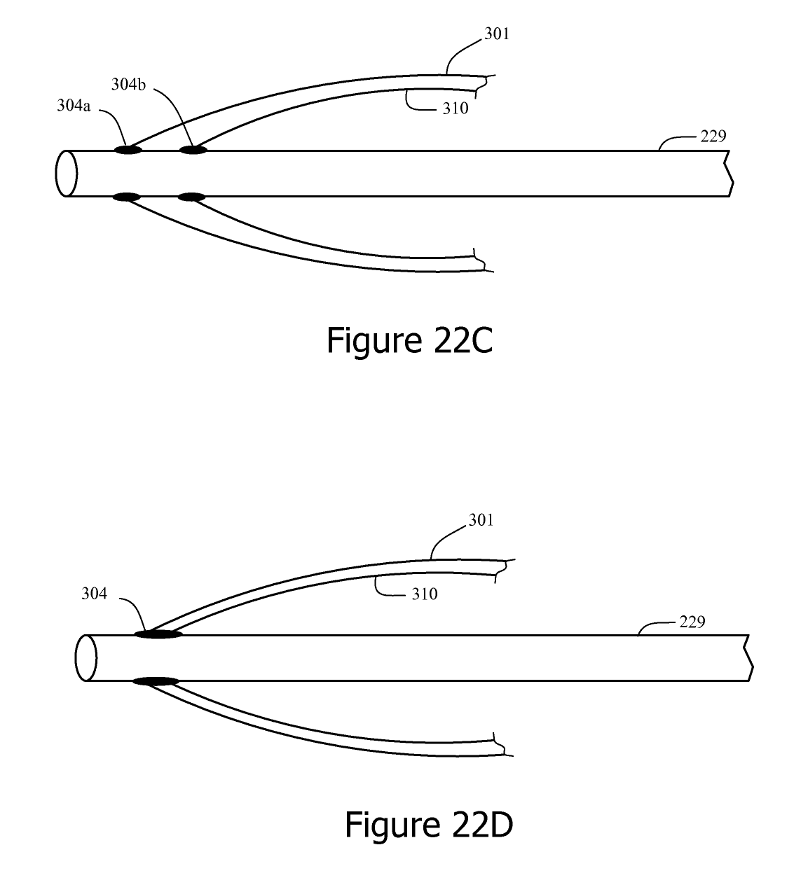

FIGS. 22A and 22B show a braid member 301 having an electrically conductive pattern 303 provided over a balloon 310 of a treatment catheter 210 in accordance embodiments of the invention. According to the embodiment illustrated in FIGS. 22A and 22B, a braid member 301 of a type described previously is affixed over a balloon 310, such as by use of an adhesive or a welding technique. In some embodiments, as is shown in FIG. 22C, two seals 304a and 304b can be created at each end of the balloon 310 on the treatment catheter's shaft 229 which bond the braid member 301 to the balloon 310. In other embodiments, as is shown in FIG. 22D, a single seal 304 can be created at each end of the balloon 310 on the treatment catheter's shaft 229 which bonds the braid member 301 to the balloon 310. Laser or heat with compression may be used to create the braid/balloon bond in accordance with these and other embodiments. In the embodiments shown in FIGS. 22A-22D, compression, tensioning, and relaxation of the braid member 301 is controlled by pressurizing and depressurizing the balloon 310.

FIG. 22A shows the balloon 310 in a non-inflated (or partially inflated) configuration, with the braid member 301 in a relaxed or tensioned state. The balloon 310 and braid member 301 shown in FIG. 22A have a substantially cylindrical shape with a length, L₁, and a diameter, D₁. In FIG. 22B, the balloon 310 is shown in an inflated configuration, with the braid member 301 in a compressed configuration. With the balloon 310 in an inflated configuration, the braid member 301 assumes a bulbous shape with a length, L₂, and a diameter, D₂, where D₂>>D₁ and L₂<<L₁.

The braid member 301 shown in FIGS. 22A and 22B is configured as a contiguous component. In some embodiments, the braid member 301 may comprise multiple components provided on the balloon 310 in a spaced-apart relationship, and that the multiple components may be electrically coupled in series or parallel, allowing for denervation therapy delivery as a single treatment element (e.g., when connected in series) or a separately controllable multi-component treatment element (e.g., when connected in parallel).

In some embodiments, the balloon 310 may incorporate a cooling fluid circulation arrangement that is fluidly coupled to one or more lumens of the treatment catheter 210. This also allows for control of internal pressure of the balloon 310 to avoid overstretching damage to the renal artery. This further allows for measurement of balloon fluid temperature to avoid overheating the artery wall and causing restenosis. Provision of a cooling fluid to the circulation arrangement of the balloon 310 facilitates controlled cooling at the braid member 301 and the wall of the renal artery in contact with the braid member 301, which serves to reduce thermal damage to non-targeted renal artery tissue.

According to other embodiments, a treatment catheter 210 may be provided with multiple balloons 310A-310n (not shown) each having a braid member 301A-310n (see, e.g., FIG. 21) provided thereon. Each of the braid members 301A-301n may comprise a conductive pattern 303A-303n that defines a portion of a spiral, such that alignment of conductive pattern portions 303A-303n across all of the braid members 301A-301n results in a spiral shaped electrode configuration. Each of the braid members 301A-301n may be individually actuated for delivering RF denervation therapy by controlling pressurization of each individual balloons 310A-310n. Provision of multiple braid members 301A-301n on multiple balloons 310A-310n provides for enhanced control and sensor feedback for each braid member 301A-301n during RF denervation therapy. It is noted that treatment catheter embodiments employing a multiplicity of individually controlled braid members 301A-301n may be used to deliver a sequential RF denervation therapy, such as by time staggered actuation of individual braid members 301A-301n, or a concurrent RF denervation therapy, such as by concurrent actuation of some or all braid members 301A-301n.

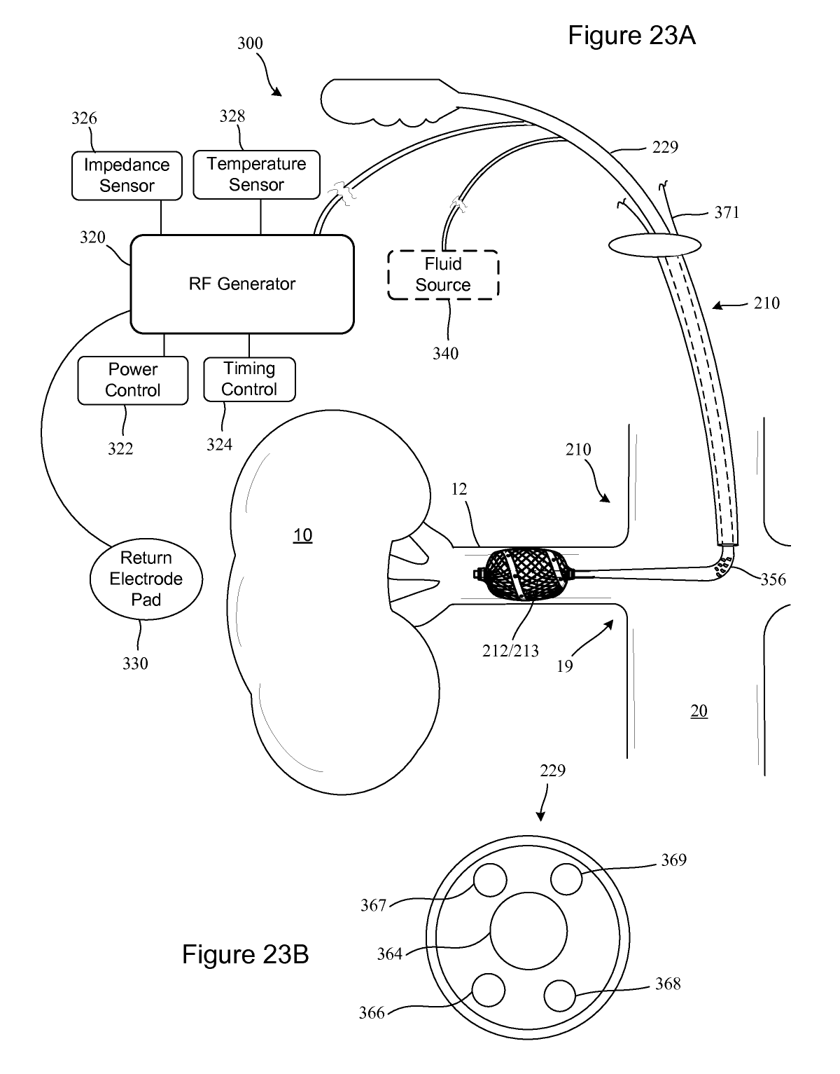

FIG. 23A shows a representative embodiment of an RF renal therapy apparatus 300 in accordance with the present invention. The apparatus 300 illustrated in FIG. 23A includes an RF generator 320 which includes power control circuitry 322 and timing control circuitry 324. The RF generator 320 is also shown to include an impedance sensor 326 and temperature measuring circuitry 328. The treatment catheter 210 includes a catheter shaft 229 that incorporates a lumen arrangement, such as that shown in FIG. 23B, configured for receiving a variety of components, including conductors, inflation fluids, pharmacological agents, actuator elements, obturators, sensors, or other components as needed or desired.

The RF generator 320 includes a return pad electrode 330 that is configured to comfortably engage the patient's back or other portion of the body near the kidneys. Radiofrequency energy produced by the RF generator 320 is coupled to the treatment section 212/213 at the distal end of the treatment catheter 210 by an appropriate conductor arrangement disposed in the lumen arrangement of the catheter's shaft 229. Renal denervation therapy using the apparatus shown in FIG. 23A is typically performed using one or more conductive element(s) of the treatment section 212/213 positioned within the renal artery and the return pad electrode 330 positioned on the patient's back, with the RF generator 320 operating in a monopolar mode.

The radiofrequency energy flows through the conductive element(s) of the treatment section 212/213, causing ionic agitation, and therefore friction in the adjacent tissue of the renal artery. This friction results in a temperature rise in the target tissues of the renal artery, including the renal nerves. After sufficient temperatures have been reached, the heat kills the target tissue within a few minutes.

In general, when renal artery tissue temperatures rise above about 113° F. (50° C.), protein is permanently damaged (including those of renal nerve fibers). For example, any mammalian tissue that is heated above about 50° C. for even 1 second is killed. If heated over about 65° C., collagen denatures and tissue shrinks. If heated over about 65° C. and up to 100° C., cell walls break and oil separates from water. Above about 100° C., tissue desiccates.

Temperature sensors 307 incorporated into the conductive element(s) of the treatment section 212/213 allow continuous monitoring of renal artery tissue temperatures, and RF generator power is automatically adjusted so that the target temperatures are achieved and maintained. An impedance sensor arrangement 326 may be used to measure and monitor electrical impedance during RF denervation therapy, and the power and timing of the RF generator 320 may be moderated based on the impedance measurements.

Depending on the power applied, duration of time the energy is applied to renal vasculature, and the resistance of renal artery tissues, temperature decreases rapidly with distance from the conductive element(s) of the treatment section 212/213, limiting lesion size and extent of damage to neighboring tissues. The size of the ablated area is determined largely by the size and shape of the conductive element(s) of the treatment section 212/213, the power applied, and the duration of time the energy is applied.

Marker bands 314 can be placed on one or multiple parts of the treatment section 212/213 to enable visualization during the procedure. Other portions of the treatment catheter 210, such as one or more portions of the catheter's shaft 229 (e.g., at the hinge mechanism 356), may include a marker band 314. The marker bands 314 may be solid or split bands of platinum or other radiopaque metal, for example. Radiopaque materials are understood to be materials capable of producing a relatively bright image on a fluoroscopy screen or another imaging technique during a medical procedure. This relatively bright image aids the user in determining specific portions of the treatment catheter 210, such as the tip of the treatment catheter 210, the treatment section 212/213, and the hinge 356, for example. The braid and/or electrode of the treatment catheter 210, according to some embodiments, can be radiopaque, and the balloon can be filled with contrast/saline if a balloon is used.

As discussed previously, the treatment catheter 210 includes a catheter shaft 229 that incorporates a lumen arrangement configured for receiving a variety of components, implements, and fluids as needed or desired. FIG. 23B shows a cross-section of a catheter shaft 229 of a treatment catheter 210 configured in accordance with embodiments of the invention.

In some embodiments, the lumen arrangement includes a lumen 364 dimensioned to receive a guide rail, such as a guide rail 202 shown in FIG. 4, or a guide wire. Other lumens, such as lumens 366, 367, 368, or 368, may be configured to receive electrical, optical, and/or fiber optic conductors, for example. One or more of lumens 366, 367, 368, and 368 may be configured to receive a pressurized fluid, such as a passive fluid (e.g., saline), a thermal transfer fluid (e.g., Freon or other fluorocarbon refrigerant, nitrous oxide, liquid nitrogen, liquid carbon dioxide), or a fluid containing a pharmacological agent (e.g., a neurotoxin or venom). One or more of lumens 366, 367, 368, and 368 may be configured to receive a shaping wire or stylet, a visualization instrument, an ultrasonic sensor/transducer, or other sensor arrangement.

In various embodiments, the apparatus 140 includes an fluid source 340 for configurations that employ one or more inflation balloons and/or thermal transfer fluid transport to and from the distal end of the treatment catheter 210. The fluid source 340, for example, may be configured to supply a pressurized fluid to one or more balloons provided at the distal end of the treatment catheter 210, as is shown in several embodiments described hereinabove. In other embodiments, the fluid source 340 may be configured to supply a thermal transfer fluid or a fluidic treatment agent to a therapy delivery element provided at the distal end of the treatment catheter 210, such as a cryotherapy or drug delivery element.

By way of example, at least two of lumens 364, 366, 367, 368, and 368 may be configured as supply and return lumens for supplying a cryogen to the distal end of the treatment catheter 210 and returning the cryogen or gas to the proximal end of the treatment catheter 210, respectively. The supply and return lumens may be coupled to a cryotube, cryoballoon, or other cryotherapy element disposed at the distal end of the treatment catheter 210. The cryogen may be circulated through the cryotherapy element via a hydraulic circuit that includes a cryogen source, supply and return lumens, and the cryotherapy element disposed at the distal end of the treatment catheter 210. In configurations that incorporate a cryotherapy element, the shaft 229 of the treatment catheter 210 is preferably lined with or otherwise incorporates insulation material(s) having appropriate thermal and mechanical characteristics suitable for a selected cryogen.

The lumen arrangement of FIG. 23B is shown for illustrative purposes only, and is not intended to limit the configuration and/or functionality of a treatment catheter 210 or a renal denervation therapy apparatus 300 implemented in accordance with the present invention. Accordingly, various lumens shown in FIG. 23B need not be incorporated in a given catheter configuration. Alternatively, lumens other than those shown in FIG. 23B may be incorporated in a given catheter configuration, including lumens formed within or on the exterior wall of the catheter's shaft 229.

As is further shown in FIG. 23A, the treatment catheter 210 may incorporate a hinge mechanism 356 built into the treatment catheter 210 proximate the treatment section 212/213. The hinge mechanism 356 is constructed to enhance user manipulation of the treatment catheter 210 when navigating around a nearly 90 degree turn from the abdominal aorta into the renal artery. It is understood that a hinge mechanism 356 may be built into other catheters and sheaths that may be used to facilitate access to the renal artery via the abdominal aorta. For example, a delivery sheath or guide catheter 371 that is used to provide renal artery access for a treatment catheter 210 of a type described herein, a guide rail (see, e.g., FIG. 4), a balloon catheter, or other device may incorporate a hinge mechanism 356.

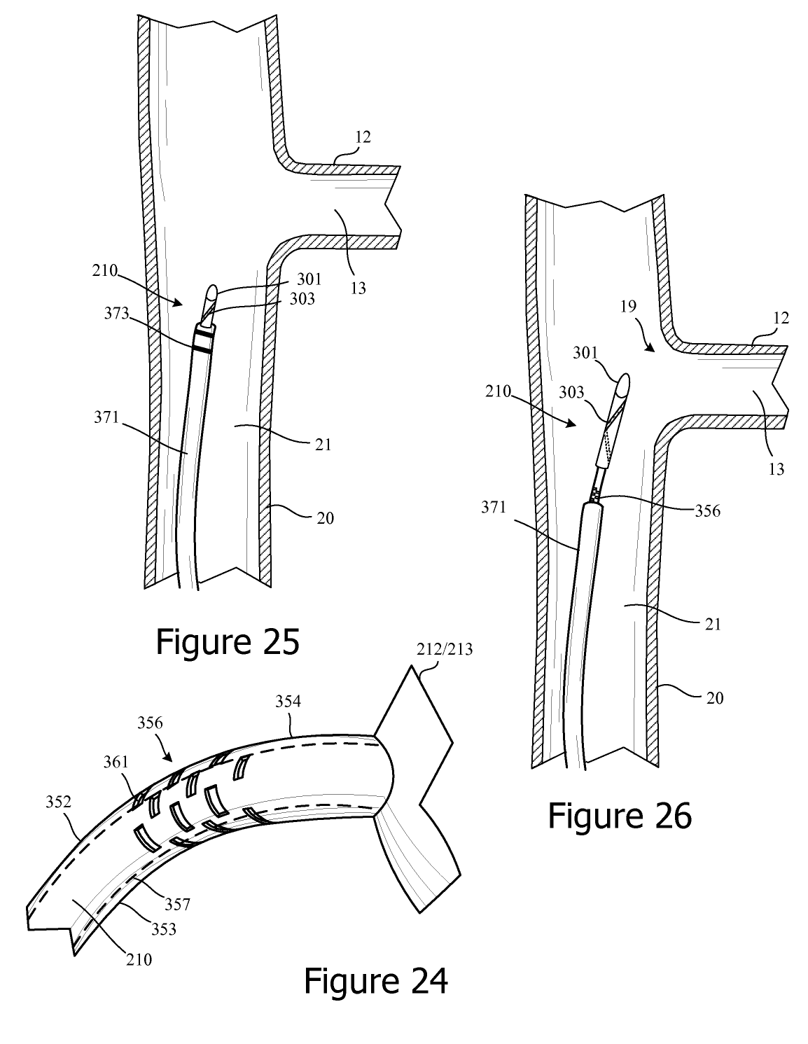

FIG. 24 illustrates a portion of the treatment catheter 210 that incorporates a hinge mechanism 356 in accordance with embodiments of the invention. The hinge mechanism 356 is provided at a location of the catheter 210 between a proximal section 352 and a distal section 354 of the catheter's shaft. The hinge mechanism 356 is preferably situated near the proximal section of the treatment element 212/213. According to various embodiments, the hinge mechanism 356 comprises a slotted tube arrangement that is configured to provide a flexible hinge point of the catheter's shaft proximate the treatment element 212/213.

The catheter's shaft may be formed to include an elongate core member 357 and a tubular member 353 disposed about a portion of the core member 357. The tubular member 353 may have a plurality of slots 361 formed therein. The slotted hinge region 356 of the catheter's shaft may be configured to have a preferential bending direction.

For example, and as shown in FIG. 24, tubular member 352 may have a plurality of slots 361 that are formed by making a pair of cuts into the wall of tubular member 361 that originate from opposite sides of tubular member 353, producing a lattice region of greater flexibility relative to the proximal and distal sections 352, 354 of the catheter's shaft. The thickness of the catheter wall at the hinge region 356 can be varied so that one side of the catheter wall is thicker than the opposite side. This difference in wall thickness alone (i.e., in embodiments devoid of slots) without or in combination with a difference in slot (void) density at the hinge region 356 provides for a preferential bending direction of the distal portion of the treatment catheter 210.

A hinge arrangement 356 constructed to provide for a preferential bending direction allows a physician to more easily and safely navigate the treatment element 212/213 to make the near 90 degree turn into the renal artery from the abdominal aorta. One or more marker bands may be incorporated at the hinge region 356 to provide visualization of this region of the catheter's shaft during deployment. Details of useful hinge arrangements that can be incorporated into embodiments of a treatment catheter 210 of the present invention or other component that facilitates access to the renal artery from the abdominal aorta are disclosed in U.S. Pat. No. 7,162,303 and U.S. Patent Publication No. 2009/0043372, which are incorporated herein by reference. It is noted that the treatment catheter 210 may incorporate a steering mechanism in addition to, or exclusion of, a hinge arrangement 356. Known steering mechanisms incorporated into steerable guide catheters may be incorporated in various embodiments of a treatment catheter 210 of the present invention.

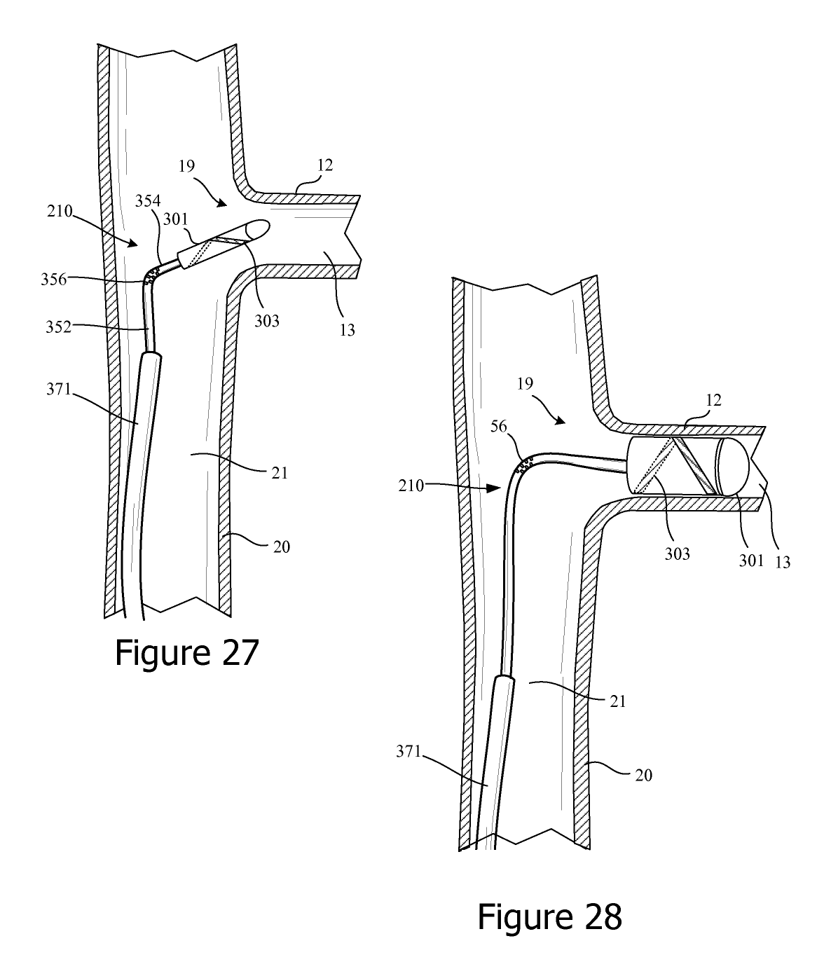

FIGS. 25-28 show a series of views of a treatment catheter 210 of the present invention at different states of deployment within aortal and renal vasculature of a patient. For purposes of illustration and not of limitation, the treatment catheter 210 shown in FIGS. 25-28 will be described as incorporating a braid member 301 comprising an electrically conductive pattern 303 and configured to deform in the manner of so-called Chinese handcuffs, as is shown in FIGS. 18A-20 and described in accompanying text.

A typical deployment procedure involves percutaneous delivery of a guide catheter 371 to an access vessel (e.g., a vascular access port into the femoral artery), via an introducer sheath (not shown), and advancement of the guide catheter 371 through access vasculature to the abdominal aorta 20 at a location inferior (or superior) to the renal artery 12. The guide catheter 371 preferably includes one or more marker bands 373 to aid in visualization of at least the distal open tip of the guide catheter 371. The guide catheter 371 may include a steering mechanism, of a type discussed above.

With the guide catheter 371 positioned near the ostium 19 of the renal artery 12, the treatment catheter 210, with the braid member 301 in a collapsed configuration, is advanced through the lumen of the guide catheter 371. Marker bands 373 may be provided on or near the braid member 301 to facilitate visualization of the braid member 301 when being advanced through the guide catheter 371 and within the renal artery 12. As is shown in FIG. 26, the braid member 301 is advanced out of the guide catheter 371, typically allowing the braid member 301 to expand somewhat upon exiting the distal open tip of the guide catheter 371. As the region of the catheter shaft comprising the hinge mechanism 356 passes out of the guide catheter 371, the distal portion 354 of the catheter shaft preferably bends relative to the proximal portion 352 of the catheter shaft in a direction dictated by the preferential bend provided by the hinge mechanism 356.

The catheter shaft may be rotated by the physician to achieve proper orientation of the braid member 301 relative to the ostium 19 of the renal artery 12. Further advancement of the braid member 301 (or retraction of the guide catheter 371) relative to the guide catheter 371 allows for an increase in bend angle at the hinge region 356, allowing the physician to safely advance the distal tip of the braid member 301 into the ostium 19 of the renal artery lumen 13. After the braid member 301 is advanced to a desired location within the renal artery 12, the actuator apparatus is manipulated by the user to compress the braid member 301.

In response to compressive force, the braid member 301 expands radially so that the conductive pattern 303 comes into close proximity or contact with the renal artery's inner wall. RF energy is coupled to the conductive pattern 303 to create a spiral lesion along the renal artery's inner wall, as described previously. After completing the RF renal denervation therapy, compression of the braid member 301 is relieved, causing the braid member 301 to relax and assume a compact shape. The braid member 301 and treatment catheter 210 are then removed from the patient's body.

Embodiments of the present invention may be implemented to provide varying degrees of denervation therapy to innervated renal vasculature. For example, embodiments of the present invention may provide for control of the extent and relative permanency of renal nerve impulse transmission interruption achieved by denervation therapy delivered using a treatment apparatus of the present invention. The extent and relative permanency of renal nerve injury may be tailored to achieve a desired reduction in sympathetic nerve activity (including a partial or complete block) and to achieve a desired degree of permanency (including temporary or irreversible injury).

The extent and permanency of renal denervation for a particular patient is dependent in large part on the type of denervation technology employed. A number of different denervation technologies have been described herein, including those that use a thermal transfer fluid (hot or cold), a pharmacological agent(s), radioactive material or seeds, or electromagnetic energy (e.g., RF, microwave, laser/light, ultrasonic). Combinations of denervation therapy apparatuses of disparate type or technology can be used together (concurrently or sequentially) to enhance the efficacy of renal denervation therapy. Renal denervation therapy apparatuses in accordance with embodiments of the present invention may be implemented to facilitate titration of a desired degree and permanency of renal sympathetic nerve activity cessation, representative examples of which are described below.

Returning to FIGS. 3B and 3C, the portion of the renal nerve 14 shown in FIGS. 3B and 3C includes bundles 14a of nerve fibers 14b each comprising axons or dendrites that originate or terminate on cell bodies or neurons located in ganglia or on the spinal cord, or in the brain. Supporting tissue structures 14c of the nerve 14 include the endoneurium (surrounding nerve axon fibers), perineurium (surrounds fiber groups to form a fascicle), and epineurium (binds fascicles into nerves), which serve to separate and support nerve fibers 14b and bundles 14a. In particular, the endoneurium, also referred to as the endoneurium tube or tubule, is a layer of delicate connective tissue that encloses the myelin sheath of a nerve fiber 14b within a fasciculus.

Major components of a neuron include the soma, which is the central part of the neuron that includes the nucleus, cellular extensions called dendrites, and axons, which are cable-like projections that carry nerve signals. The axon terminal contains synapses, which are specialized structures where neurotransmitter chemicals are released in order to communicate with target tissues. The axons of many neurons of the peripheral nervous system are sheathed in myelin, which is formed by a type of glial cell known as Schwann cells. The myelinating Schwann cells are wrapped around the axon, leaving the axolemma relatively uncovered at regularly spaced nodes, called nodes of Ranvier. Myelination of axons enables an especially rapid mode of electrical impulse propagation called saltation.

In some embodiments, a treatment apparatus of the present invention may be implemented to deliver denervation therapy that causes transient and reversible injury to renal nerve fibers 14b. In other embodiments, a treatment apparatus of the present invention may be implemented to deliver denervation therapy that causes more severe injury to renal nerve fibers 14b, which may be reversible if the therapy is terminated in a timely manner. In preferred embodiments, a treatment apparatus of the present invention may be implemented to deliver denervation therapy that causes severe and irreversible injury to renal nerve fibers 14b, resulting in permanent cessation of renal sympathetic nerve activity. For example, a treatment apparatus may be implemented to deliver a denervation therapy that disrupts nerve fiber morphology to a degree sufficient to physically separate the endoneurium tube of the nerve fiber 14b, which can prevent regeneration and re-innervation processes.

By way of example, and in accordance with Seddon's classification as is known in the art, a treatment apparatus of the present invention may be implemented to deliver a denervation therapy that interrupts conduction of nerve impulses along the renal nerve fibers 14b by imparting damage to the renal nerve fibers 14b consistent with neruapraxia. Neurapraxia describes nerve damage in which there is no disruption of the nerve fiber 14b or its sheath. In this case, there is an interruption in conduction of the nerve impulse down the nerve fiber, with recovery taking place within hours to months without true regeneration, as Wallerian degeneration does not occur. Wallerian degeneration refers to a process in which the part of the axon separated from the neuron's cell nucleus degenerates. This process is also known as anterograde degeneration. Neurapraxia is the mildest form of nerve injury that may be imparted to renal nerve fibers 14b by use of a treatment apparatus according to embodiments of the present invention.

A treatment apparatus may be implemented to interrupt conduction of nerve impulses along the renal nerve fibers 14b by imparting damage to the renal nerve fibers consistent with axonotmesis. Axonotmesis involves loss of the relative continuity of the axon of a nerve fiber and its covering of myelin, but preservation of the connective tissue framework of the nerve fiber. In this case, the encapsulating support tissue 14c of the nerve fiber 14b are preserved. Because axonal continuity is lost, Wallerian degeneration occurs. Recovery from axonotmesis occurs only through regeneration of the axons, a process requiring time on the order of several weeks or months. Electrically, the nerve fiber 14b shows rapid and complete degeneration. Regeneration and re-innervation may occur as long as the endoneural tubes are intact.

A treatment apparatus may be implemented to interrupt conduction of nerve impulses along the renal nerve fibers 14b by imparting damage to the renal nerve fibers 14b consistent with neurotmesis. Neurotmesis, according to Seddon's classification, is the most serious nerve injury in the scheme. In this type of injury, both the nerve fiber 14b and the nerve sheath are disrupted. While partial recovery may occur, complete recovery is not possible. Neurotmesis involves loss of continuity of the axon and the encapsulating connective tissue 14c, resulting in a complete loss of autonomic function, in the case of renal nerve fibers 14b. If the nerve fiber 14b has been completely divided, axonal regeneration causes a neuroma to form in the proximal stump.

A more stratified classification of neurotmesis nerve damage may be found by reference to the Sunderland System as is known in the art. The Sunderland System defines five degrees of nerve damage, the first two of which correspond closely with neurapraxia and axonotmesis of Seddon's classification. The latter three Sunderland System classifications describe different levels of neurotmesis nerve damage.

The first and second degrees of nerve injury in the Sunderland system are analogous to Seddon's neurapraxia and axonotmesis, respectively. Third degree nerve injury, according to the Sunderland System, involves disruption of the endoneurium, with the epineurium and perineurium remaining intact. Recovery may range from poor to complete depending on the degree of intrafascicular fibrosis. A fourth degree nerve injury involves interruption of all neural and supporting elements, with the epineurium remaining intact. The nerve is usually enlarged. Fifth degree nerve injury involves complete transection of the nerve fiber 14b with loss of continuity.

As discussed above in accordance with various embodiments, denervation therapy may be delivered to innervated renal vasculature using a treatment arrangement that incorporates a cryotherapy element. Renal denervation therapy may be controlled to achieve a desired degree of attenuation in renal nerve activity in accordance with embodiments of the present invention. For example, renal nerve fiber regeneration and re-innervation may be permanently compromised by applying cryogenic therapy to innervated renal vasculature at a sufficiently low temperature to allow ice crystals to form inside nerve fibers 14b. Formation of ice crystals inside nerve fibers 14b of innervated renal arterial tissue and renal ganglia tears the nerve cells apart, and physically disrupts or separates the endoneurium tube, which can prevent regeneration and re-innervation processes. Delivery of cryogenic therapy to renal nerves 14 at a sufficiently low temperature in accordance with embodiments of the present invention can cause necrosis of renal nerve fibers 14b, resulting in a permanent and irreversible loss of the conductive function of renal nerve fibers 14b.

In general, embodiments of a treatment catheter of the present invention may be implemented to deliver cryogenic therapy to cause renal denervation at therapeutic temperatures ranging between approximately 0° C. and approximately −180° C. For example, embodiments of a treatment catheter may be implemented to deliver cryogenic therapy to cause renal denervation with temperatures at the renal nerves ranging from approximately 0° C. to approximately −30° C. at the higher end, and to about −140° C. to −180° C. at the lower end. Less robust renal nerve damage is likely for temperatures approaching and greater than 0° C., and more robust acute renal denervation is likely for temperatures approaching and less than −30° C., for example, down to −120 C. to −180 C. These therapeutic temperature ranges may be determined empirically for a patient, a patient population, or by use of human or other mammalian studies.

It has been found that delivering cryotherapy to the renal artery and the renal ganglia at a sufficiently low temperature with freeze/thaw cycling allows ice crystals to form inside nerve fibers 14b and disrupt renal nerve function and morphology. For example, achieving therapeutic temperatures that range from −30° C. to +10° C. at a renal nerve for treatment times of 30 seconds to 4 minutes and thaw times of about 1 to 2 minutes has been found to cause acute renal denervation in at least some of the renal nerves in a porcine model.

The representative embodiments described below are directed to apparatuses that can deliver cryogenic therapy to renal vasculature at specified therapeutic temperatures or temperature ranges, causing varying degrees of nerve fiber degradation. As was discussed above, therapeutic temperature ranges achieved by treatment catheters of the present invention may be determined using non-human mammalian studies. The therapeutic temperatures and degrees of induced renal nerve damage described in the context of the following embodiments are based largely on cryoanalgesia studies performed on rabbits (see, e.g., L. Zhou et al., Mechanism Research of Cryoanalgesia, Neurological Research, Vol. 17, pp. 307-311 (1995)), but may generally be applicable for human renal vasculature. As is discussed below, the therapeutic temperatures and degrees of induced renal nerve damage may vary somewhat or significantly from those described in the context of the following embodiments based on a number of factors, including the design of the cryotherapy apparatus, duration of cryotherapy, and the magnitude of mechanical disruption of nerve fiber structure that can be achieved by subjecting renal nerves to freeze/thaw cycling, among others.

In accordance with various embodiments, a treatment catheter of the present invention may be implemented to deliver cryogenic therapy to cause a minimum level of renal nerve damage. Cooling renal nerve fibers to a therapeutic temperature ranging between about 0° C. and about −20° C. is believed sufficient to temporarily block some or all renal sympathetic nerve activity and cause a minimum degree of renal nerve damage, consistent with neurapraxia for example. Freezing renal nerves to a therapeutic temperature of −20° C. or higher may not cause a permanent change in renal nerve function or morphology. At therapeutic temperatures of −20° C. or higher, slight edema and myelin swelling may occur in some of the renal nerve fibers, but these conditions may be resolved after thawing.

In other embodiments, cooling renal nerve fibers to a therapeutic temperature ranging between about −20° C. and about −60° C. is believed sufficient to block all renal sympathetic nerve activity and cause an intermediate degree of renal nerve damage, consistent with axonotmesis (and possibly some degree of neurotmesis for lower temperatures of the −20° C. and −60° C. range), for example. Cooling renal nerves to a therapeutic temperature of −60° C. may cause freezing degeneration and loss of renal nerve conductive function, but may not result in a permanent change in renal nerve function or morphology. However, renal nerve regeneration is substantially slowed (e.g., on the order of 90 days). At a therapeutic temperature of −60° C., the frozen renal nerve is likely to demonstrate edema with thickening and loosening of the myelin sheaths and irregular swelling of axons, with Schwann cells likely remaining intact.

In further embodiments, cooling renal nerve fibers to a therapeutic temperature ranging between about −60° C. and about −100° C. is believed sufficient to block all renal sympathetic nerve activity and cause an intermediate to a high degree of renal nerve damage, consistent with neurotmesis, for example. Cooling renal nerves to a therapeutic temperature of −100° C., for example, causes swelling, thickening, and distortion in a large percentage of axons. Exposing renal nerves to a therapeutic temperature of −100° C. likely causes splitting or focal necrosis of myelin sheaths, and microfilament, microtubular, and mitochondrial edema. However, at a therapeutic temperature of −100° C., degenerated renal nerves may retain their basal membranes, allowing for complete recovery over time. Although substantially slowed (e.g., on the order of 180 days), renal nerve regeneration may occur and be complete.

In accordance with other embodiments, cooling renal nerve fibers to a therapeutic temperature of between about −140° C. and about −180° C. is believed sufficient to block all renal sympathetic nerve activity and cause a high degree of renal nerve damage, consistent with neurotmesis for example. Application of therapeutic temperatures ranging between about −140° C. and about −180° C. to renal nerve fibers causes immediate necrosis, with destruction of basal membranes (resulting in loss of basal laminea scaffolding needed for complete regeneration). At these low temperatures, axoplasmic splitting, axoplasmic necrosis, and myelin sheath disruption and distortion is likely to occur in most renal nerve fibers. Proliferation of collagen fibers is also likely to occur, which restricts renal nerve regeneration.

It is believed that exposing renal nerves to a therapeutic temperature of about −140° C. or lower causes permanent, irreversible damage to the renal nerve fibers, thereby causing permanent and irreversible termination of renal sympathetic nerve activity. For some patients, exposing renal nerves to a therapeutic temperature ranging between about −120° C. and about −140° C. may be sufficient to provide similar permanent and irreversible damage to the renal nerve fibers, thereby causing permanent and irreversible cessation of renal sympathetic nerve activity. In other patients, it may be sufficient to expose renal nerves to a therapeutic temperature of at least −30° C. in order to provide a desired degree of renal sympathetic nerve activity cessation.

In preferred embodiments, it is desirable that the cryogen used to deliver cryotherapy to renal vasculature be capable of freezing target tissue so that nerve fibers innervating the renal artery are irreversibly injured, such that nerve conduction along the treated renal nerve fibers is permanently terminated. Suitable cryogens include those capable of cooling renal nerve fibers and renal ganglia to temperatures of at least about −120° C. or lower, preferably to temperatures of at least about −130° C. or lower, and more preferably to temperatures of at least about −140° C. or lower. It is understood that use of cryogens that provide for cooling of renal nerve fibers and renal ganglia to temperatures of at least about −30° C. may effect termination of renal sympathetic nerve activity with varying degrees of permanency.

The temperature ranges and associated degrees of induced renal nerve damage described above are provided for non-limiting illustrative purposes. Actual therapeutic temperatures and magnitudes of resulting nerve injury may vary somewhat or significantly from those described herein, and be impacted by a number of factors, including patient-specific factors (e.g., the patient's unique renal vasculature and sympathetic nervous system characteristics), therapy duration, frequency and duration of freeze/thaw cycling, structural characteristics of the cryotherapy catheter/element, type of cryogen used, and method of delivering cryotherapy, among others.

It is believed that higher degrees of renal nerve injury may be achieved by subjecting renal nerves to both cryotherapy and freeze/thaw cycling when compared to delivering cryotherapy without employing freeze/thaw cycling. Implementing freeze/thaw cycling as part of cryotherapy delivery to renal nerves may result in achieving a desired degree of renal sympathetic nerve activity attenuation (e.g., termination) and permanency (e.g., irreversible) at therapeutic temperatures higher than those discussed above. Various thermal cycling parameters may be selected for, or modified during, renal denervation cryotherapy to achieve a desired level of renal nerve damage, such parameters including the number of freeze/thaw cycles, high and low temperature limits for a given freeze/thaw cycle, the rate of temperature change for a given freeze/thaw cycle, and the duration of a given freeze/thaw cycle, for example. As was previously discussed, these therapeutic temperature ranges and associated degrees of induced renal nerve damage may be determined empirically for a particular patient or population of patients, or by use of human or other mammalian studies.

The foregoing description of the various embodiments of the invention has been presented for the purposes of illustration and description. It is not intended to be exhaustive or to limit the invention to the precise form disclosed. Many modifications and variations are possible in light of the above teaching. For example, the devices and techniques disclosed herein may be employed in vasculature of the body other than renal vasculature, such as coronary and peripheral vessels and structures. It is intended that the scope of the invention be limited not by this detailed description, but rather by the claims appended hereto.

What is claimed is:

1. An apparatus, comprising:
a catheter comprising a proximal end, a distal end, and a length sufficient to access at least a renal artery relative to a percutaneous access location of the patient;
a braid provided at a distal end of the catheter and dimensioned for deployment within the renal artery, the braid comprising:
a material having a resiliency that facilitates deployment of the braid into the renal artery from the abdominal aorta;
a proximal end, a distal end, a length, and a diameter;
an electrically conductive pattern having a substantially helical shape that completes at least one revolution of the braid, the electrically conductive pattern configured to electrically couple with a radiofrequency generator;
insulating portions defining regions of the braid devoid of the electrically conductive pattern;
the braid configured to decrease in length and increase in diameter in response to axial compression, and to increase in length and decrease in diameter in response to axial tensioning or relaxation; and
an actuator coupled to the braid and actuatable at the proximal end of the catheter, the actuator coupled to at least one of the proximal and distal ends of the braid and configured to selectively extend and compress the braid longitudinally, the electrically conductive pattern of the braid urged towards and away from an inner wall of the renal artery in response to braid compression and relaxation, respectively;
wherein denervation therapy is delivered to the renal artery with the braid in compression and by energizing the electrically conductive pattern by the generator.

2. The apparatus of claim 1, wherein the braid material comprises a plurality of voids that define a perfusion arrangement which facilitates arterial blood flow through the braid for cooling an inner wall of the renal artery.

3. The apparatus of claim 1, wherein different regions of the electrically conductive pattern are sequentially compressible by the actuator and electrically activatable for forming a series of burn spots which collectively form a spiral lesion.

4. The apparatus of claim 1, comprising a sensor arrangement provided at or coupled to a plurality of locations of the braid and configured to sense temperature or impedance at each of the plurality of braid locations.

5. The apparatus of claim 1, comprising a plurality of temperature sensors provided at longitudinally spaced locations of the electrically conductive pattern, each of the plurality of temperature sensors configured to sense a temperature at one of the longitudinally spaced locations, thereby providing a temperature profile of the electrically conductive pattern of the braid.

6. The apparatus of claim 1, comprising at least one sensor provided at or coupled to the braid, wherein the generator is configured to automatically control power delivery to the braid in response to a signal produced by the at least one sensor during denervation therapy delivery.

7. The apparatus of claim 1, wherein the braid comprises a plurality of braid sections each comprising a segment of the substantially helical shaped electrically conductive pattern.

8. The apparatus of claim 7, wherein each of the plurality of braid sections is coupled to one of a plurality of actuator members for providing independent actuation thereof.

9. The apparatus of claim 7, wherein each of the plurality of braid sections is coupled to one of a plurality of electrical conductor arrangements of the catheter for providing independent electrical activation and deactivation thereof.

10. The apparatus of claim 1, wherein the braid comprises filaments that are woven together in a crossed alternating configuration.

11. The apparatus of claim 1, wherein the material of the braid comprises an electrically insulating material.

12. The apparatus of claim 1, wherein the material of the braid comprises a polymeric material.

13. The apparatus of claim 1, wherein one of the proximal end and the distal end of the braid is positionally fixed to the distal end of the catheter, and the other of the proximal end and the distal end of the braid is movably affixed on the catheter and coupled to the actuator.

14. The apparatus of claim 1, wherein each of the proximal end and the distal end of the braid is movably affixed to the distal end of the catheter and coupled to the actuator.

15. An apparatus, comprising:
a catheter comprising a proximal end, a distal end, and a length sufficient to access at least a renal artery relative to a percutaneous access location of the patient;
a balloon disposed at the distal end of the catheter and fluidly coupled to a lumen of the catheter, the balloon configured for deployment within the renal artery and to receive a thermal transfer fluid via the lumen; and
a braid provided on a surface of the balloon, the braid comprising:
a resilient material;
a proximal end, a distal end, a length, and a diameter;
an electrically conductive pattern having a substantially helical shape that completes at least one revolution of the braid, the electrically conductive pattern configured to electrically couple with a radiofrequency generator for delivering renal denervation therapy; and
insulating portions defining regions of the braid devoid of the electrically conductive pattern.

16. The apparatus of claim 15, wherein the balloon comprises a circulation arrangement through which the thermal transfer fluid circulates for cooling the inner wall of the renal artery during delivery of renal denervation therapy.

17. The apparatus of claim 15, wherein different regions of the electrically conductive pattern are sequentially activatable for forming a series of burn spots which collectively form a spiral lesion.

18. The apparatus of claim 15, comprising a sensor arrangement provided at or coupled to a plurality of locations of the braid and configured to sense temperature or impedance at each of the plurality of braid locations.

19. The apparatus of claim 15, comprising a plurality of temperature sensors provided at longitudinally spaced locations of the electrically conductive pattern, each of the plurality of temperature sensors configured to sense a temperature at one of the longitudinally spaced locations, thereby providing a temperature profile of the electrically conductive pattern of the braid.

20. The apparatus of claim 15, comprising at least one sensor provided at or coupled to the braid, wherein the generator is configured to automatically control power delivery to the braid in response to a signal produced by the at least one sensor during denervation therapy delivery.

21. The apparatus of claim 15, wherein the braid comprises a plurality of braid sections each comprising a segment of the substantially helical shaped electrically conductive pattern.

22. The apparatus of claim 21, wherein each of the plurality of braid sections is coupled to one of a plurality of electrical conductor arrangements of the catheter for providing independent electrical activation and deactivation thereof.

23. The apparatus of claim 15, wherein the braid material comprises at least one of:
filaments that are woven together in a crossed alternating configuration;
an electrically insulating material; and
a polymeric material.

24. A method, comprising:
extending a braid disposed at a distal end of a catheter longitudinally for deployment of the braid within a renal artery of a patient;
compressing the braid longitudinally so that an electrically conductive pattern of the braid is urged towards an inner wall of the renal artery;
energizing the electrically conductive pattern to create a lesion in the artery having a substantially spiral shape;
cooling the braid while energizing the electrically conductive pattern to cool the inner wall of the artery; and
extending the braid longitudinally subsequent to energizing the electrically conductive pattern for removal of the braid from the patient's renal artery.
